(19) United States
(12) Patent Application Publication
(10) Pub. No.: US 2025/0188113 A1
(43) Pub. Date: Jun. 12, 2025

(54) HIGH PURITY NON-ANIMAL DERIVED TUDCA

(71) Applicant: SANDHILL ONE, LLC, West Palm Beach, FL (US)

(72) Inventors: J Gregory REID, West Palm Beach, FL (US); Daniel John GANLEY, West Palm Beach, FL (US); Jayachandra P REDDY, West Palm Beach, FL (US)

(73) Assignee: SANDHILL ONE, LLC, West Palm Beach, FL (US)

(21) Appl. No.: 18/704,437

(22) PCT Filed: Nov. 1, 2022
(86) PCT No.: PCT/US2022/079081

§ 371 Date: Apr. 24, 2024

Related U.S. Application Data
(60) Provisional application No. 63/274,534, filed on Nov. 2, 2021.
(60) Provisional application No. 63/390,239, filed on Jul. 18, 2022.

(51) Int. Cl. C07J 41/00; A61K 31/575

(52) U.S. Cl. CPC C07J 41/0061; A61K 31/575

(57) ABSTRACT

Methods of making cholic acid derivatives, particularly TUDCA, preferably from non-animal sources, having exceptional purity and therapeutic utility.

### RELATED APPLICATIONS

This application claims priority to U.S. Ser. No. 63/274,534, filed Nov. 2, 2021, and to U.S. Ser. No. 63/390,239, filed Jul. 18, 2022. The contents of these applications are incorporated by reference as if fully set forth herein.

### FIELD OF THE INVENTION

The present invention relates generally to cholic acid derivatives, particularly TUDCA, having exceptional purity and therapeutic utility, preferably derived from non-animal sources, and to methods and intermediates used for making same.

### BACKGROUND OF THE INVENTION

Cholic acid and its derivatives find utility in numerous medical applications and research initiatives. Cholic acid itself, sold under the brand name Cholbam®, is approved for use as a treatment for children and adults with bile acid synthesis disorders due to single enzyme defects, and for peroxisomal disorders (such as Zellweger syndrome). 7-Ketolithocholic acid has been examined for its effect on endogenous bile acid synthesis, biliary cholesterol saturation, and its possible role as a precursor of chenodeoxycholic acid and ursodeoxycholic acid. See Salen et al. Gasteroenterology, 1982; 83:341-7. Ursodeoxycholic acid (a/k/a UDCA or ursodiol), sold under the brand name URSO 250® and URSO ForteR tablets, is approved for the treatment of patients with primary biliary cirrhosis (PBC). More recently, obeticholic acid, sold under the brand name Ocaliva, was approved for the treatment of PBC in combination with UDCA in adults with an inadequate response to UDCA, or as monotherapy in adults unable to tolerate UDCA.

In spite of this significant medical interest in cholic acid derivatives, methods of synthesizing the derivatives remain a cumbersome inefficient process, with numerous processes being proposed. Fantin et al. Steroids, 1993 November; 58:524-526, discloses the preparation of 7α-, 12α-, 12β-hydroxy and 7α-, 12α- and 7α-, 12β-dihydroxy-3-ketocholanoic acids by protecting the 3-keto group as dimethyl ketal and subsequent reduction with sodium borohydride of the corresponding 7- and 12-oxo functionalities. WO 2017/079062 A1 by Galvin reports a method of preparing obeticholic acid by direct alkylation at the C-6 position of 7-keto lithocholic acid (KLCA). He et al., Steroids, 2018 December; 140:173-178, discloses a synthetic route of producing ursodeoxycholic acid (UDCA) and obeticholic acid (OCA) through multiple reactions from cheap and readily-available cholic acid. Wang et al., Steroids 157 (2020) 108600, similarly report a synthetic route of producing ursodeoxycholic acid (UDCA) through multiple reactions from commercially available bisnoralcohol (BA).

Commercially available preparations containing bile acids such as tauroursodeoxycholic acid (TUDCA) are derived exclusively from animal corpses such as cows and sheep, which pose the threat of contamination by pathogens such as prions and other toxins. In addition, even though bile acids from animal sources are typically purified in order to exclude impurities, in practice, such purified compositions contain a mixture of bile acids due to the difficulty separating closely related analogs and isomers. The United States Pharmacopoeia explicitly permits CDCA in UDCA, and Rajevic (1998) report several commercially available compositions of UDCA of animal origin, all containing some chenodeoxycholic acid (CDCA). Rajevic M and Betto P, J. Liq. Chrom. & Rel. Technol., 21 (18), 2821-2830 (1998).

TUDCA is similarly always contaminated by related impurities, commonly derived from the UDCA used to produce the TUDCA, or the process of making the TUDCA itself. EP 1 985 622 A1, for example, reports a method of manufacturing TUDCA and a “pure” TUDCA that contains less than 0.2% taurine, less than 0.5% UDCA, and less than 0.3% of any other impurities, having a total TUDCA content greater than 98.5%.

What is needed are more efficient processes for making cholic acid derivatives, especially TUDCA. A particular need exists for the production of non-animal derived cholic acid derivatives, and processes that eliminate the production of harmful analogs and isomers in TUDCA.

### SUMMARY OF INVENTION

The inventors have developed, for the first time, methods that enable the production of non-animal derived sources of TUDCA, having a 813C signature corresponding to plant-derived material. Thus, in a first principal embodiment the invention provides a compound selected from a taurine conjugate of ursodeoxycholic acid of formula I:

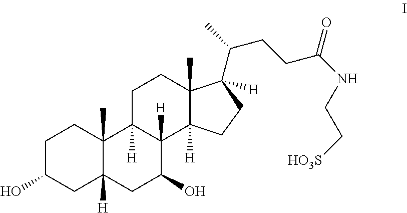

and its salts comprising a δ¹³C value corresponding to a plant derived molecule, preferably comprising less than −20‰, −22.5‰, or −25‰ δ¹³C relative to VPDB.

The invention also provides TUDCA of animal and nonanimal origin having an exceptional purity profile, essentially devoid of UDCA, taurine, and other impurities. Thus, in a second principal embodiment the invention provides a compound selected from a taurine conjugate of ursodeoxycholic acid of formula I:

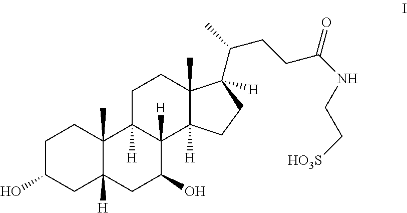

and its salts comprising an impurity profile characterized by: (a) less than 0.1%, 0.05%, 0.03%, or 0.01% of UDCA; (b) less than 0.1%, 0.05%, 0.03%, or 0.01% of taurine; (c) less than 1.0%, 0.50%, 0.30% or 0.10% of 5α-TUDCA, and optionally greater than 0.05% 5α-TUDCA; (d) less than 0.20%, 0.10%, 0.05% of TCDCA; and/or (e) a combination thereof.

The invention further provides novel crystalline forms of TUDCA and to novel salts of TUDCA. Thus, in a third principal embodiment the invention provides a crystalline plant-derived taurine conjugate of ursodeoxycholic acid of formula I (TUDCA):

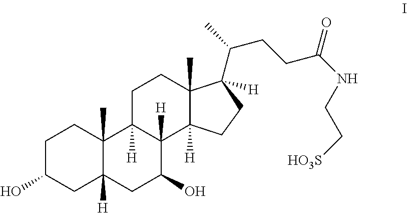

comprising: (a) an XRPD pattern corresponding to Form A TUDCA or Form L TUDCA; and (b) a δ¹³C value corresponding to a plant derived molecule, preferably comprising less than −20‰, −22.5‰, or −25‰ δ¹³C relative to VPDB.

In a fourth principal embodiment the invention provides a crystalline taurine conjugate of ursodeoxycholic acid of formula I (TUDCA):

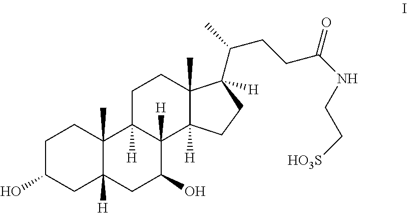

comprising an XRPD pattern corresponding to Form L TUDCA.

In a fifth principal embodiment the invention provides a salt of a taurine conjugate of ursodeoxycholic acid of formula I:

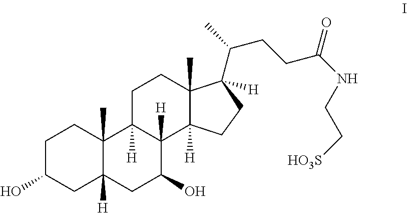

selected from the group consisting of arginine TUDCA, histidine TUDCA, and lysine TUDCA.

The invention further provides methods of making TUDCA having exceptional purity from contamination by the stereoisomeric impurities 3 β-hydroxysteroids and 7α-hydroxysteroids. Thus, in a sixth principal embodiment the invention provides a method of producing the compound of the first or second principal embodiment that goes through a TDKCA intermediate comprising: (a) contacting the TDKCA with a 3α-hydroxysteroid dehydrogenase to stereo-selectively reduce the TDKCA to a 3α-hydroxy intermediate, and contacting the 3α-hydroxy intermediate with a 7β-hydroxysteroid dehydrogenase to stereo-selectively reduce the 3α-hydroxy intermediate to TUDCA; (b) contacting the TDKCA with a 7β-hydroxysteroid dehydrogenase to stereo-selectively reduce the TDKCA to a 7β-hydroxy intermediate, and contacting the 7β-hydroxy intermediate with a 3 α-hydroxysteroid dehydrogenase to stereo-selectively reduce the 7β-hydroxy intermediate to TUDCA; or (c) simultaneously contacting the TDKCA with a 3α-hydroxysteroid dehydrogenase and a 7β-hydroxysteroid dehydrogenase to stereo-selectively reduce the TDKCA to TUDCA. In a preferred embodiment the TDKCA is first provided in an isolated state.

In a seventh principal embodiment the invention provides a method of making TUDCA or a salt thereof comprising: (a) (i) contacting 3,7-DKCA with a 3α-hydroxysteroid dehydrogenase to stereo-selectively reduce the 3,7-KDCA to a 3α-hydroxy intermediate, and contacting the 3a-hydroxy intermediate with a 7β-hydroxysteroid dehydrogenase to stereo-selectively reduce the 3a-hydroxy intermediate to UDCA; (ii) contacting the 3,7-DKCA with a 7β-hydroxysteroid dehydrogenase to stereo-selectively reduce the 3,7-DKCA to a 7β-hydroxy intermediate, and contacting the 7β-hydroxy intermediate with a 3α-hydroxysteroid dehydrogenase to stereo-selectively reduce the 7β-hydroxy intermediate to UDCA; or (iii) simultaneously contacting the 3,7-DKCA with a 3α-hydroxysteroid dehydrogenase and a 7β-hydroxysteroid dehydrogenase to stereo-selectively reduce the 3,7-DKCA to UDCA, and (b) conjugating the UDCA with taurine to form TUDCA, wherein the 3,7-DKCA is optionally provided as or derived from an ethylenediamine salt of 3,7-DKCA (optionally Pattern 6-D), a tert-butylamine salt of 3,7-DKCA (optionally Pattern 9-A), or a diisopropylamine salt of 3,7-DKCA (optionally Pattern 10-A). In a preferred embodiment, the 3,7-DKCA is first provided in an isolated state.

The invention further relates to the novel intermediates made when practicing the methods of the current invention. Thus, in an eighth principal embodiment the invention provides 3a-Hydroxy-7-oxo-5β-cholanoyltaurine or a salt thereof.

In a ninth principal embodiment the invention provides 7β-Hydroxy-3-oxo-5β-cholanoyltaurine or a salt thereof.

In a tenth principal embodiment the invention provides 3,7-Oxo-5β-cholanoyltaurine or a salt thereof.

In an eleventh principal embodiment the invention provides an ethylenediamine salt of 3,7-DKCA.

In a twelfth principal embodiment the invention provides a tert-butylamine salt of 3,7-DKCA.

In a thirteenth principal embodiment the invention provides a diisopropylamine salt of 3,7-DKCA.

Additional advantages of the invention are set forth in part in the description that follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE FIGURES

The accompanying drawings, which are incorporated in and constitute a part of this specification, illustrate several embodiments of the invention and together with the description serve to explain the principles of the invention.

FIG. 1 is an XRPD diffractogram of solid Pattern 1-A from L-arginine and IPA:MeOH (7:3 vol.) and commercial TUDCA derived from plant sources (L-arginine TUDCA).

FIG. 2 is a DSC thermogram of Pattern 1-A (L-arginine TUDCA).

FIG. 3 is an XRPD diffractogram for solid Pattern 5-A from L-lysine, ACN:MeOH (1:1 vol.), and commercial TUDCA derived from plant sources (L-lysine TUDCA).

FIG. 4 is a long-scan XRPD pattern for solid Pattern 6-A, from L-histidine and THF:IPA (4:6 vol.) and commercial TUDCA derived from plant sources (L-histidine TUDCA).

FIG. 5 is a DSC thermogram of solid Pattern 6-A (L-histidine TUDCA).

FIG. 6 is a XRPD diffractogram of commercial grade solid Pattern A TUDCA derived from plant sources.

FIG. 7 is a DSC thermogram of solid Pattern A TUDCA.

FIG. 8 is a XRPD diffractogram of solid Pattern L TUDCA derived from commercial grade TUDCA Pattern A plant sources.

FIG. 9 is a DSC thermogram of solid Pattern L TUDCA derived from commercial grade TUDCA Pattern A.

FIG. 10 is an HPLC chromatogram of the 3,7-DKCA starting material used in Example 13. The peak at 5.482 min is the 5-α stereoisomer.

FIG. 11 is an HPLC chromatogram of the 3,7-DKCA produced by the method of Example 13. The 5-α stereoisomeric impurity is not detected.

FIG. 12 is a high-resolution XRPD diffractogram of scaled-up Pattern 9-A from salt formation with tert-butylamine in ethanol.

FIG. 13 is a high-resolution XRPD diffractogram of scaled-up Pattern 6-D from salt screening with ethylenediamine in IPA:water (9:1 vol.).

FIG. 14 is a high-resolution XRPD diffractogram of scaled-up Pattern 10-A from salt screening with diisopropylamine in MIBK/heptane.

FIG. 15 is an HPLC chromatogram of tert-butylamine salt of 3,7-DKCA produced substantially according to the 3-picoline solvent hydrogenation and tert-butylamine crystallization methods described herein.

### DETAILED DESCRIPTION OF THE INVENTION

### Definitions and Use of Terms

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

As used in the specification and claims, the singular forms a, an, and the include plural references unless the context clearly dictates otherwise. For example, the term “a specification” refers to one or more specifications for use in the presently disclosed methods and systems. “A hydrocarbon” includes mixtures of two or more such hydrocarbons, and the like.

When the term “any” is used herein, in reference to the lack of contaminants or impurities, it will be understood that the term includes zero % but that some contaminants or impurities can also be present, but always below the limit of detection (typically <0.05% or <0.03%).

The word “or” or like terms as used herein means any one member of a particular list and also includes any combination of members of that list. Thus, when a list comprises “A, B, or C,” the list could alternatively be written as comprising “A, B, C, or a combination thereof,” or as comprising “A, B, C, A+B, A+C, B+C, or A+B+C.”

As used in this specification and in the claims which follow, the word “comprise” and variations of the word, such as “comprising” and “comprises,” means “including but not limited to,” and is not intended to exclude, for example, other additives, components, integers or steps. When an element is described as comprising one or a plurality of components, steps or conditions, it will be understood that the element can also be described as “consisting of” or “consisting essentially of” the component, step or condition, or the plurality of components, steps or conditions.

When ranges are expressed herein by specifying alternative upper and lower limits of the range, it will be understood that the endpoints can be combined in any manner that is mathematically feasible. Thus, for example, a range of from 50 or 80 to 100 or 70 can alternatively be expressed as a series of ranges of from 50 to 100, from 50 to 70, and from 80 to 100. When a series of upper bounds and lower bounds are related using the phase and/or, it will be understood that the upper bounds can be unlimited by the lower bonds or combined with the lower bounds, and vice versa. Thus, for example, a range of greater than 40% and/or less than 80% includes ranges of greater than 40%, less than 80%, and greater than 40% but less than 80%.

When used herein the term “about” will compensate for variability allowed for in the pharmaceutical industry and inherent in pharmaceutical products. In one embodiment the term allows for any variation within 5% of the recited specification or standard. In one embodiment the term allows for any variation within 10% of the recited specification or standard.

UDCA, or ursodeoxycholic acid, is represented by the following chemical structure:

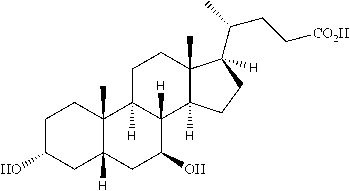

Using the methods of the current invention, UDCA can be derived from plant and animal sources, and combinations of plant and animal sources. When UDCA is expressed without specifying its source, it will be understood to encompass UDCA from any source, and with any δ¹³C content.

Tauroursodeoxycholic acid, or TUDCA, has the following chemical structure:

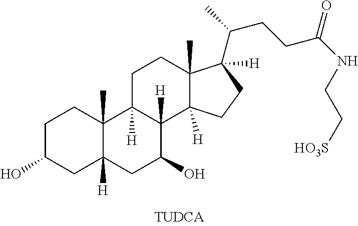

TUDCA can exist as a free acid or a salt. When expressed without specifying the free acid or salt form, the term “TUDCA” or “tauroursodeoxycholic acid” will be understood to encompass both the free acid and its salts. Using the methods of the current invention, TUDCA can be derived from plant and animal sources, and combinations of plant and animal sources. When TUDCA is expressed without specifying its source, it will be understood to encompass TUDCA from any source, and with any δ¹³C content.

TDKCA, or 3,7-Oxo-5β-cholanoyltaurine, has the following chemical structure:

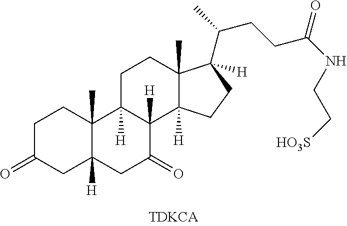

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use or use in a dietary supplement. “Pharmaceutically acceptable salts” means salts that are pharmaceutically acceptable, as defined above, and which possess the desired pharmacological or chemical activity.

“Fossil carbon percentage” means the percentage of carbon atoms in a molecule derived from “synthetic” (petrochemical) sources. “Fossil/animal” means derived exclusively from fossil sources, derived exclusively from animal sources, or derived from fossil and animal sources.

“δ¹³C value” is an isotopic measurement of the delta notation of ¹³C. δ¹³C values are expressed as a per mil (‰) deviation, e.g. per one thousand, from an internationally accepted PDB standard (originally a carbonate from the Pee Dee Belemnite formation in South Carolina but more commonly today Vienna Pee Dee Belemnite (VPDB)). δ¹³C values are determined using the following formula:

$${δ}^{13} C = ( {( {δ}^{13} C / {δ}^{12} C )}_{sample} - {( {δ}^{13} C / {δ}^{12} C )}_{PDB} ) / ( {( {δ}^{13} C / {δ}^{12} C )}_{PDB} ) * 1000$$

By “plant sources” are meant any source, which may be defined as a plant such as for example trees, shrubs, herbs, grasses, ferns, mosses, flowers, vegetables, and weeds, as well as compounds derived from plants such as phytosterols, and phytosterol derivatives. The plant can be a C3 plant, a C4 plant, or a combination of both.

The term “plant derived” refers to a molecule comprising a δ¹³C value corresponding to a plant derived molecule or a mixed fossil/animal and plant derived molecule, comprising a majority of plant-derived carbons. A plant derived molecule can thus be characterized as having greater than 50%, 75%, 90%, 95%, 98%, or 99% plant derived carbons, with the remaining carbons (if any) derived from fossil/animal resources.

By “C3 plants” are meant plants that do not have photosynthetic adaptations to reduce photorespiration. This includes plants such as rice, wheat, soybeans, most fruits, most vegetables and all trees.

By “C4 plants” are meant plants where the light-dependent reactions and the Calvin cycle are physically separated and where the light-dependent reactions occur in the mesophyll cells and the Calvin cycle occurs in bundle-sheath cells. This includes plants such as crabgrass, sugarcane, sorghum and corn.

### Discussion of Principal Embodiments

The invention can be defined based on several principal embodiments which can be combined in any manner physically and mathematically possible to create additional principal embodiments.

A first principal embodiment the invention provides a compound selected from a taurine conjugate of ursodeoxycholic acid of formula I:

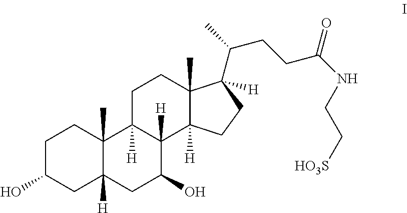

and its salts comprising a δ¹³C value corresponding to a plant derived molecule, preferably comprising less than −20‰, −22.5‰, or −25‰ δ¹³C relative to VPDB.

A second principal embodiment the invention provides a compound selected from a taurine conjugate of ursodeoxycholic acid of formula I:

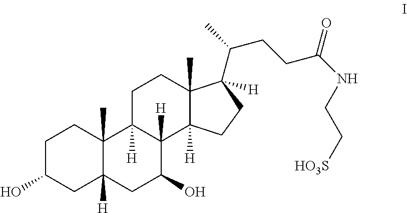

and its salts comprising an impurity profile characterized by: (a) less than 0.1%, 0.05%, 0.03%, or 0.01% of UDCA; (b) less than 0.1%, 0.05%, 0.03%, or 0.01% of taurine; (c) less than 1.0%, 0.50%, 0.30% or 0.10% of 5α-TUDCA, and optionally greater than 0.05% 5α-TUDCA; (d) less than 0.20%, 0.10%, 0.05% of TCDCA; and/or (e) a combination thereof.

In a third principal embodiment the invention provides a crystalline plant-derived taurine conjugate of ursodeoxycholic acid of formula I (TUDCA):

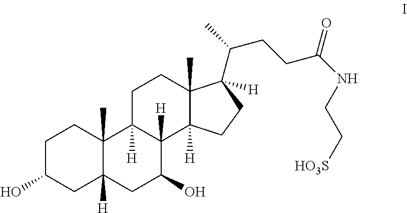

comprising: (a) an XRPD pattern corresponding to Form A TUDCA or Form L TUDCA; and (b) a δ¹³C value corresponding to a plant derived molecule, preferably comprising less than −20‰, −22.5‰, or −25‰ δ¹³C relative to VPDB.

In a fourth principal embodiment the invention provides a crystalline taurine conjugate of ursodeoxycholic acid of formula I (TUDCA):

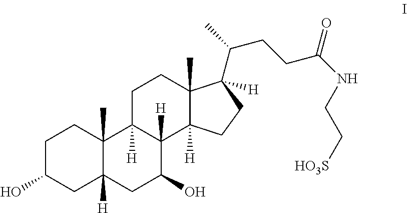

comprising an XRPD pattern corresponding to Form L TUDCA.

In a fifth principal embodiment the invention provides a salt of a taurine conjugate of ursodeoxycholic acid of formula I:

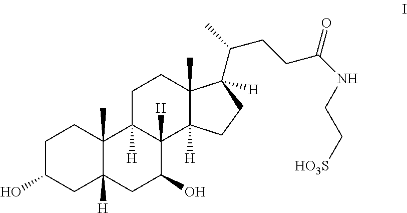

selected from the group consisting of arginine TUDCA, histidine TUDCA, and lysine TUDCA.

In a sixth principal embodiment the invention provides a method of producing the compound of the first or second principal embodiment that goes through a TDKCA intermediate comprising: (a) contacting the TDKCA with a 3α-hydroxysteroid dehydrogenase to stereo-selectively reduce the TDKCA to a 3α-hydroxy intermediate, and contacting the 3α-hydroxy intermediate with a 7β-hydroxysteroid dehydrogenase to stereo-selectively reduce the 3α-hydroxy intermediate to TUDCA; (b) contacting the TDKCA with a 7β-hydroxysteroid dehydrogenase to stereo-selectively reduce the TDKCA to a 7β-hydroxy intermediate, and contacting the 7β-hydroxy intermediate with a 3α-hydroxysteroid dehydrogenase to stereo-selectively reduce the 7β-hydroxy intermediate to TUDCA; or (c) simultaneously contacting the TDKCA with a 3α-hydroxysteroid dehydrogenase and a 7β-hydroxysteroid dehydrogenase to stereo-selectively reduce the TDKCA to TUDCA. In a preferred embodiment, the TDKCA is provided in an isolated state.

In a seventh principal embodiment the invention provides a method of making TUDCA or a salt thereof comprising: (a) (i) contacting 3,7-DKCA with a 3α-hydroxysteroid dehydrogenase to stereo-selectively reduce the 3,7-KDCA to a 3α-hydroxy intermediate, and contacting the 3α-hydroxy intermediate with a 7β-hydroxysteroid dehydrogenase to stereo-selectively reduce the 3α-hydroxy intermediate to UDCA; (ii) contacting the 3,7-DKCA with a 7β-hydroxysteroid dehydrogenase to stereo-selectively reduce the 3,7-DKCA to a 7β-hydroxy intermediate, and contacting the 7β-hydroxy intermediate with a 3α-hydroxysteroid dehydrogenase to stereo-selectively reduce the 7β-hydroxy intermediate to UDCA; or (iii) simultaneously contacting the 3,7-DKCA with a 3α-hydroxysteroid dehydrogenase and a 7β-hydroxysteroid dehydrogenase to stereo-selectively reduce the 3,7-DKCA to UDCA, and (b) conjugating the UDCA with taurine to form TUDCA, wherein the 3,7-DKCA is optionally provided as or derived from an ethylenediamine salt of 3,7-DKCA (optionally Pattern 6-D), a tert-butylamine salt of 3,7-DKCA (optionally Pattern 9-A), or a diisopropylamine salt of 3,7-DKCA (optionally Pattern 10-A). In a preferred embodiment, the 3,7-DKCA is provided in an isolated state.

In an eighth principal embodiment the invention provides 3α-Hydroxy-7-oxo-5β-cholanoyltaurine or a salt thereof. In one embodiment the 3α-Hydroxy-7-oxo-5β-cholanoyltaurine in its free form. In another embodiment, the 3α-Hydroxy-7-oxo-5β-cholanoyltaurine is present in the substantial absence of any salt forms, such that there is no meaningful interference from the salt forms when the 3α-Hydroxy-7-oxo-5β-cholanoyltaurine is subjected to ketoreduction using the ketoreductases described herein.

In a ninth principal embodiment the invention provides 7β-Hydroxy-3-oxo-5β-cholanoyltaurine or a salt thereof. In one embodiment the 7β-Hydroxy-3-oxo-5β-cholanoyltaurine in its free form. In another embodiment, the 7β-Hydroxy-3-oxo-5β-cholanoyltaurine is present in the substantial absence of any salt forms, such that there is no meaningful interference from the salt forms when the 7β-Hydroxy-3-oxo-5β-cholanoyltaurine is subjected to ketoreduction using the ketoreductases described herein.

In a tenth principal embodiment the invention provides 3,7-Oxo-5β-cholanoyltaurine or a salt thereof. In one embodiment the 3,7-Oxo-5β-cholanoyltaurine in its free form. In another embodiment, the 3,7-Oxo-5β-cholanoyltaurine is present in the substantial absence of any salt forms, such that there is no meaningful interference from the salt forms when the 3,7-Oxo-5β-cholanoyltaurine is subjected to ketoreduction using the ketoreductases described herein.

In an eleventh principal embodiment the invention provides an ethylenediamine salt of 3,7-DKCA.

In a twelfth principal embodiment the invention provides a tert-butylamine salt of 3,7-DKCA.

In a thirteenth principal embodiment the invention provides a diisopropylamine salt of 3,7-DKCA.

### Discussion of Subembodiments

The invention can further be understood with reference to various subembodiments which can modify any of the principal embodiments. These subembodiments can be combined in any manner that is both mathematically and physically possible to create additional subembodiments, which in turn can modify any of the principal embodiments. For example, any of the subembodiments requiring a plant-derived TUDCA can be used to further modify the TUDCA embodiments not limited by plant origin. In like manner, any of the purity subembodiments can be used to further modify an embodiment with broader purity allowances.

In any of the purity embodiments or subembodiments of the current invention, it will be understood that some measure of impurity can also be present (even if non-detectable by current analytical techniques), or that none can be present, and that when the impurity is present, it is preferably present in an amount greater than 0.001% or 0.005%. Thus:
whenever a compound is stated to contain less than a certain percentage of UDCA, it will be understood that the compound can also be expressed in alternative embodiments as containing greater than 0.001% or 0.005% UDCA;
whenever a compound is stated to contain less than a certain percentage of taurine, it will be understood that the compound can also be expressed in alternative embodiments as containing greater than 0.001% or 0.005% taurine;
whenever a compound is stated to contain less than a certain percentage of 3β-hydroxysteroids or 3β-TUDCA, it will be understood that the compound can also be expressed in alternative embodiments as containing greater than 0.001% or 0.005% 3β-hydroxysteroids or 3β-TUDCA;
whenever a compound is stated to contain less than a certain percentage of 5α-steroids or 5α-TUDCA, it will be understood that the compound can also be expressed in alternative embodiments as containing greater than 0.001% or 0.005% 5α-steroids or 5α-TUDCA;
whenever a compound is stated to contain less than a certain percentage of 7a-hydroxysteroids or TCDCA, it will be understood that the compound can also be expressed in alternative embodiments as containing greater than 0.001% or 0.005% 7α-hydroxysteroids or TCDCA;

Similarly, when TUDCA is referred to herein as plant derived, it will be understood that the TUDCA will preferably comprising less than −20‰, −22.5‰, or −25‰ SVC relative to VPDB, most preferably less than −20‰ SVC relative to VPDB.

In one subembodiment, any of the TUDCA principal embodiments (i.e. principal embodiments 1-6) are modified to provide a plant derived TUDCA comprising: (a) less than 5%, 3%, 1%, 0.5%, 0.1%, 0.05%, 0.03%, or 0.01% of UDCA; (b) less than 5%, 3%, 1%, 0.5%, 0.1%, 0.05%, 0.03%, or 0.01% of taurine; (c) less than 5%, 3%, 1%, 0.5%, 0.1%, 0.05%, 0.03%, or 0.01% of any 3β-hydroxysteroids; (d) less than 5%, 3%, 1%, 0.5%, 0.1%, 0.05%, 0.03%, or 0.01% of any 5α-steroids; or (e) less than 5%, 3%, 1%, 0.5%, 0.1%, 0.05%, 0.03%, or 0.01% of any 7a-hydroxysteroids,

In another subembodiment, any of the TUDCA principal embodiments are modified to provide plant derived TUDCA comprising: (a) less than 1% of UDCA; (b) less than 1% of taurine; (c) less than 1% of any 3β-hydroxysteroids; (d) less than 1% of any 5α-steroids; or (e) less than 1% of any 7α-hydroxysteroids.

In another embodiment, any of the TUDCA principal embodiments are modified to provide plant derived TUDCA comprising: (a) less than 0.1% of UDCA; (b) less than 0.1% of taurine; (c) less than 0.1% of any 3β-hydroxysteroids; (d) less than 0.1% of any 5α-steroids; or (e) less than 0.1% of any 7α-hydroxysteroids.

In another embodiment, any of the TUDCA principal embodiments are modified to provide plant derived TUDCA comprising less than 5%, 3%, 1%, 0.5%, 0.1%, 0.05%, 0.03%, or 0.01% of any UDCA.

In another embodiment, any of the TUDCA principal embodiments are modified to provide plant derived TUDCA comprising less than 5%, 3%, 1%, 0.5%, 0.1%, 0.05%, 0.03%, or 0.01% of any taurine.

In another embodiment, any of the TUDCA principal embodiments are modified to provide plant derived TUDCA comprising less than 5%, 3%, 1%, 0.5%, 0.1%, 0.05%, 0.03%, or 0.01% of any 3β-hydroxysteroids.

In another embodiment, any of the TUDCA principal embodiments are modified to provide plant derived TUDCA comprising less than 5%, 3%, 1%, 0.5%, 0.1%, 0.05%, 0.03%, or 0.01% of any 5α-steroids.

In another embodiment, any of the TUDCA principal embodiments are modified to provide plant derived TUDCA comprising less than 5%, 3%, 1%, 0.5%, 0.1%, 0.05%, 0.03%, or 0.01% of any 5α-TUDCA.

In another embodiment, any of the TUDCA principal embodiments are modified to provide plant derived TUDCA comprising less than 5%, 3%, 1%, 0.5%, 0.1%, 0.05%, 0.03%, or 0.01% of any 7α-hydroxysteroids.

In another embodiment, any of the TUDCA principal embodiments are modified to provide plant derived TUDCA comprising less than 5%, 3%, 1%, 0.5%, 0.1%, 0.05%, 0.03%, or 0.01% of any TCDCA.

In another embodiment, any of the TUDCA principal embodiments are modified to provide plant derived TUDCA comprising: (a) less than 5%, 3%, 1%, 0.5%, 0.1%, 0.05%, 0.03%, or 0.01% of UDCA; (b) less than 5%, 3%, 1%, 0.5%, 0.1%, 0.05%, 0.03%, or 0.01% of taurine; (c) less than 5%, 3%, 1%, 0.5%, 0.1%, 0.05%, 0.03%, or 0.01% of any 3 O-hydroxysteroids; (d) less than 5%, 3%, 1%, 0.5%, 0.1%, 0.05%, 0.03%, or 0.01% of any 5α-steroids; and (e) less than 5%, 3%, 1%, 0.5%, 0.1%, 0.05%, 0.03%, or 0.01% of any 7α-hydroxysteroids.

In another embodiment, any of the TUDCA principal embodiments are modified to provide plant derived TUDCA comprising: (a) less than 1% of UDCA; (b) less than 1% of taurine; (c) less than 1% of any 3 β-hydroxysteroids; (d) less than 1% of any 5α-steroids; and (e) less than 1% of any 7α-hydroxysteroids.

In another embodiment, any of the TUDCA principal embodiments are modified to provide plant derived TUDCA comprising: (a) less than 0.1% of UDCA; (b) less than 0.1% of taurine; (c) less than 0.1% of any 3β-hydroxysteroids; (d) less than 0.1% of any 5α-steroids; and (e) less than 0.1% of any 7α-hydroxysteroids.

In another embodiment, any of the TUDCA principal embodiments are modified to provide TUDCA comprising less than 0.1%, 0.05%, 0.03%, or 0.01% of UDCA.

In another embodiment, any of the TUDCA principal embodiments are modified to provide TUDCA comprising less than 0.1%, 0.05%, 0.03%, or 0.01% of taurine.

In another embodiment, any of the TUDCA principal embodiments are modified to provide TUDCA comprising less than 0.1%, 0.05%, 0.03%, or 0.01% of any UDCA, further comprising less than 1% or 0.5% of impurities selected from starting materials, by-products, intermediates, and degradation products.

In another embodiment, any of the TUDCA principal embodiments are modified to provide TUDCA comprising less than 0.1%, 0.05%, 0.03%, or 0.01% of taurine, further comprising less than 1% or 0.5% of impurities selected from starting materials, by-products, intermediates, and degradation products.

In another embodiment, any of the TUDCA principal embodiments are modified to provide TUDCA comprising: (a) less than 0.1%, 0.05%, 0.03%, or 0.01% of UDCA, and (b) less than 0.1%, 0.05%, 0.03%, or 0.01% of taurine, further comprising less than 1% or 0.5% of impurities selected from starting materials, by-products, intermediates, and degradation products.

In another embodiment, any of the TUDCA principal embodiments are modified to comprise: (i) less than 5%, 3%, 1%, 0.5%, 0.1%, 0.05%, 0.03%, or 0.01% of any 3-keto, 7-hydroxysteroids; (ii) less than 5%, 3%, 1%, 0.5%, 0.1%, 0.05%, 0.03%, or 0.01% of any 3-hydroxy, 7-ketosteroids; (iii) less than 5%, 3%, 1%, 0.5%, 0.1%, 0.05%, 0.03%, or 0.01% of TDKCA; (iv) less than 5%, 3%, 1%, 0.5%, 0.1%, 0.05%, 0.03%, or 0.01% of any 5α-steroids; and (v) combinations thereof.

Particularly preferred TUDCA in any of the embodiments of the current invention is free from any 7α-hydroxysteroids.

Particularly preferred TUDCA in any of the embodiments of the current invention is free from any UDCA.

The TUDCA in any of the embodiments of the current invention preferably comprises less than 3%, 2%, or 1% of impurities selected from starting materials, by-products, intermediates, and degradation products.

The TUDCA in any of the embodiments of the current invention is optionally present in an isolated state.

The inventive compounds derive in one embodiment from the ability to control/eliminate the production of 3p-hydroxysteroids and 7α-hydroxysteroids using the ketoreductases of the present invention. The strategy also permits the elimination of UDCA impurities in the final product since UDCA is not used in the synthesis, and a drastic reduction in the potential for taurine contamination since taurine is conjugated to the steroid far upstream of the final product isolation.

Therefore, in still further embodiments the invention provides TUDCA made by a process that goes through a TDKCA intermediate, comprising: (a) contacting the TDKCA with a 3α-hydroxysteroid dehydrogenase to stereo-selectively reduce the TKDCA to a 3α-hydroxy intermediate, and contacting the 3α-hydroxy intermediate with a 7β-hydroxysteroid dehydrogenase to stereo-selectively reduce the 3α-hydroxy intermediate to TUDCA; (b) contacting the TDKCA with a 7β-hydroxysteroid dehydrogenase to stereo-selectively reduce the TDKCA to a 7β-hydroxy intermediate, and contacting the 7β-hydroxy intermediate with a 3α-hydroxysteroid dehydrogenase to stereo-selectively reduce the 7β-hydroxy intermediate to TUDCA; or (c) simultaneously contacting the TDKCA with a 3α-hydroxysteroid dehydrogenase and a 7β-hydroxysteroid dehydrogenase to stereo-selectively reduce the TDKCA to TUDCA.

Subsequent reduction by 3α-hydroxysteroid dehydrogenases and 7β-hydroxysteroid dehydrogenases produce novel intermediates 8 and/or 9, depending on the sequence of reduction, as depicted in the following scheme:

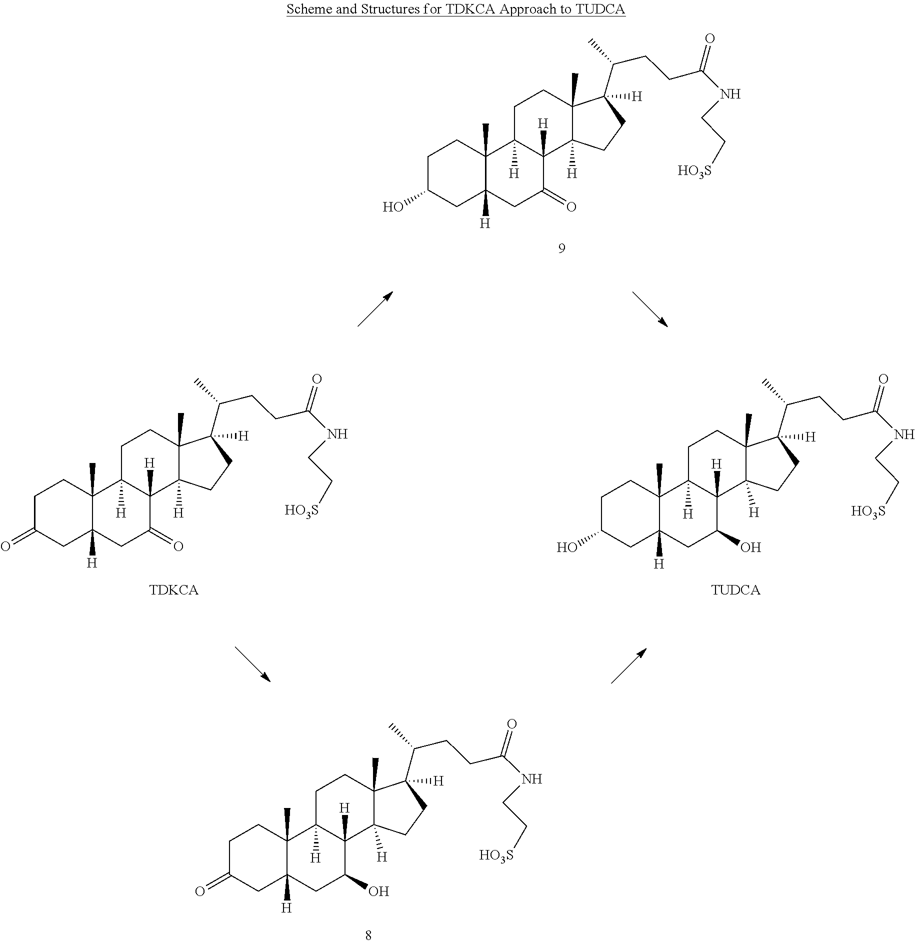

where:
TDKCA=Tauro-3,7-dioxo-5β-cholanic acid, or 3,7-dioxo-5β-cholanoyltaurine
Compound 9=3α-Hydroxy,7-oxo-5β-cholanoyltaurine
Compound 8=7β-Hydroxy,3-oxo-5β-cholanoyltaurine
TUDCA=Tauroursodeoxylcholic acid, or 3α,7β-dihydroxy-5β-cholanoyltaurine

The inventive compounds also derive from the novel 3,7-DKCA crystalline salts disclosed herein.

Thus, in one embodiment the TDKCA is derived from the ethylenediamine salt of 3,7-DKCA, preferably a crystalline form defined by Pattern 6-D.

In another embodiment the TDKCA is derived from the tert-butylamine salt of 3,7-DKCA, preferably a crystalline form defined by Pattern 9-A.

In still another embodiment, the TDKCA is derived from the diisopropylamine salt of 3,7-DKCA, preferably a crystalline form defined by Pattern 10-A.

Thus, the methods of the current invention may further include:
providing a precursor compound selected from:
   an ethylenediamine salt of 3,7-DKCA (optionally Pattern 6-D),
   a tert-butylamine salt of 3,7-DKCA (optionally Pattern 9-A), or
   a diisopropylamine salt of 3,7-DKCA (optionally Pattern 10-A), and
conjugating the compound with taurine to form TDKCA.
   Of course, it will be understood that the salt can first be removed from the 3,7-DKCA before taurine conjugation or from the TDKCA before ketoreduction.

It will also be understood that the process can be performed without going through a TDKCA intermediate using any of the 3,7-DKCA salts as precursor compounds to UDCA, and conjugating taurine directly to UDCA. Thus, in yet another embodiment the invention provides a method of making TUDCA or a salt thereof comprising: (a) (i) contacting 3,7-DKCA with a 3α-hydroxysteroid dehydrogenase to stereo-selectively reduce the 3,7-KDCA to a 3α-hydroxy intermediate, and contacting the 3α-hydroxy intermediate with a 7β-hydroxysteroid dehydrogenase to stereo-selectively reduce the 3α-hydroxy intermediate to UDCA; (ii) contacting the 3,7-DKCA with a 7β-hydroxysteroid dehydrogenase to stereo-selectively reduce the 3,7-DKCA to a 7β-hydroxy intermediate, and contacting the 7β-hydroxy intermediate with a 3α-hydroxysteroid dehydrogenase to stereo-selectively reduce the 7β-hydroxy intermediate to UDCA; or (iii) simultaneously contacting the 3,7-DKCA with a 3α-hydroxysteroid dehydrogenase and a 7β-hydroxysteroid dehydrogenase to stereo-selectively reduce the 3,7-DKCA to UDCA, and (b) conjugating the UDCA with taurine to form TUDCA, wherein the 3,7-DKCA is provided as or derived from an ethylenediamine salt of 3,7-DKCA (optionally Pattern 6-D), a tert-butylamine salt of 3,7-DKCA (optionally Pattern 9-A), or a diisopropylamine salt of 3,7-DKCA (optionally Pattern 10-A). Once again, when going through a salt precursor, it will be understood that the salt can first be removed from the 3,7-DKCA before ketoreduction.

### Taurine Conjugation

In the synthesis of TUDCA from UDCA and taurine, or the synthesis of TDKCA from 3,7-DKCA and taurine, the UDCA or 3,7-DKCA is first converted to an acylating agent, which is subsequently reacted with taurine to form TUDCA or TDKCA having the following chemical structure, where the UDCA or 3,7-DKCA and taurine are bound through an amide bond:

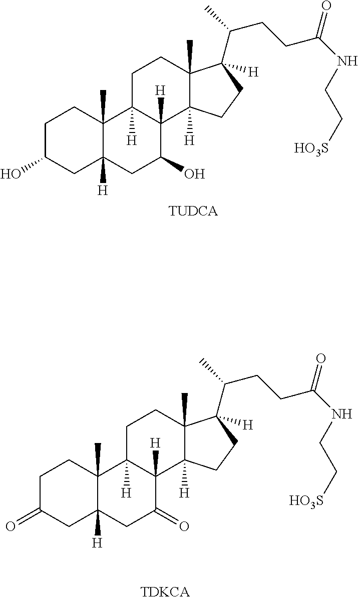

The UDCA or 3,7-DKCA can be converted to various acylating agents suitable for the claimed reaction, as taught generally by Morrison & Boyd, Organic Chemistry, 6th Edition (Benjamin-Cummings Publishing Company) and John Welch, Organic Chemistry, Synthesis, in Encyclopedia of Physical Science and Technology (Third Edition), 2003. Common acylating agents to which the UDCA can be converted, which are suitable for the production of amides like TUDCA, are acid anhydrides (including alkoxycarbonyl mixed anhydrides and phosphonic anhydrides), N-hydroxysuccinimidyl esters, N-hydroxybenzotriazole esters, imidazolides, phenyl esters and acyl halides (a/k/a acid halides).

The reaction with UDCA proceeds according to the following pathway:

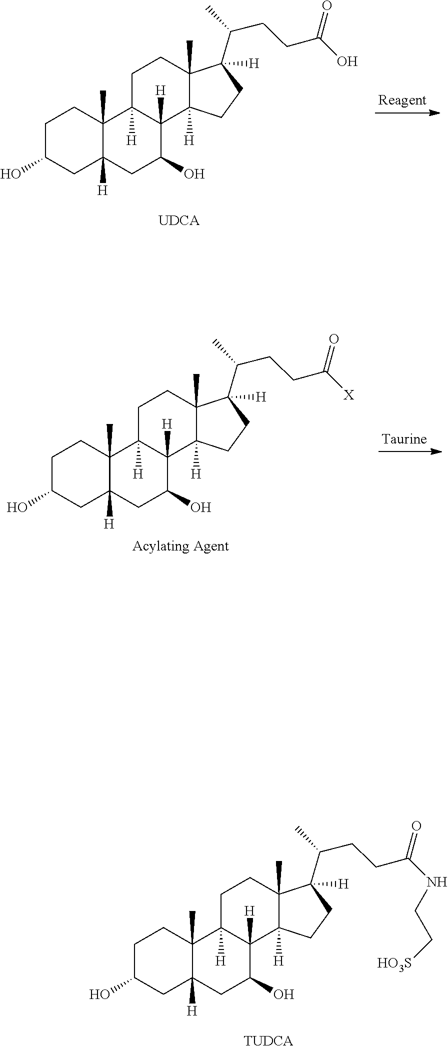

Table 1 lists suitable reagents and exemplifying references for converting UDCA or DKCA (or an ester thereof) to an acylating agent, although these reagents are in no way meant to be limiting, but simply exemplary of the numerous chemical pathways well-known to those of skill in the art:

TABLE 1

| X | Reagent | References |
|---|---|---|
| Cl | SOCl₂, ClCOCOCl, COCl₂ | 1, 2 |
| Br | SOBr₂, TPP/NBS | 1, 2 |
| Im | Im₂CO | 1, 2 |
| EtOCO₂ | EtOCOCl | 1, 2 |
| (CH₃)₃CCO₂ | (CH₃)₃CCOCl | 1, 2 |
| (MeO)₂P(O)O | (MeO)₂P(O)Cl | 2 |
| OC(NHiPr)═NiPr | Diisopropylcarbodiimide (DIC) | 1, 2 |
| OBt | DIC + HOBt | 1 |
| OSu | DIC + HOSu | 1 |
| O(1-methylpyridinium-2- | 2-Chloro-1-methylpyridinium | 1 |
| yl) | iodide | |
| OP⁺(py)₃ | (Py)₃P⁺Br (PF₆)⁻ | 1 |

TPP = triphenylphosphine; NBS = N-bromosuccinimde; Im = imidazol-1-yl; Et = ethyl; Me = methyl; iPr = isopropyl; Bt = benzotriazol-1-yl; Su = succinimid-1-yl; py = pyrrolidin-1-yl

where Reference 1 is Montalbetti and Falque, Tetrahedron, 2005, vol 61, p. 10827-10852, and Reference 2 is March's Advanced Organic Chemistry, Smith and March, 6^{th} Ed., 2007, p. 1427-1439.

Thus, in various subembodiments, the UDCA or 3,7-DKCA is converted to an acylating agent, which is subsequently reacted with taurine to form TUDCA or TDKCA or a salt thereof. In further embodiments, the UDCA or 3,7-DKCA is contacted with means for converting the 24-carboxylic acid or ester group on UDCA or 3,7-DKCA to a derivative that can act as an acylating agent, and reacting the derivative with taurine to form TUDCA or TDKCA or a salt thereof. In these subembodiments, the structure corresponding to the means would be ethyl chloroformate, as specifically described in the examples.

### Novel Salts, Hydrates, and Crystal Forms

When any compound is referenced herein, either by itself, in combination with other ingredients, or in a chemical or biological process, it will be understood that the compound can be present in or used as an isolated form. By isolated form is meant that the compound is preferably present as a solid, and that it is substantially free of any compounds other than the recited compound (i.e. <10%, 5%, 3%, or 1% other compounds).

Any of the TUDCA of the current invention, whether or not plant derived, can be present in the form of a salt, with arginine TUDCA, histidine TUDCA, and lysine TUDCA preferred. In like manner, the TUDCA can be present as a free acid, preferably a crystalline free acid having an XRPD pattern corresponding to Form A TUDCA or Form L TUDCA.

When the TUDCA is provided as Form A, the compound preferably has an X-ray powder diffraction pattern comprising at least one, three, or five peaks, in terms of 20, selected from the group consisting of 5.19, 10.31, 10.49, 19.08, 20.83, 22.03, 23.26, 23.58, 24.89, and 31.09±0.2°, preferably 1, 2, or 3 peaks, in terms of 20, selected from the group consisting of 5.19, 10.31, 19.08, and 31.09±0.2°. Alternatively, the compound can have an X-ray powder diffraction pattern substantially as depicted in FIG. 6.

When the TUDCA is provided as Form L, the compound preferably has an X-ray powder diffraction pattern comprising at one or two peaks, in terms of 20, selected from the group consisting of 4.59 and 19.610±0.2°, optionally in combination with one or any combination of 15.11, 17.56, 18.41, and 21.38°±0.2°. Alternatively, the compound can have an X-ray powder diffraction pattern substantially as depicted in FIG. 8.

When the TUDCA is provided as arginine TUDCA, the compound will preferably be crystalline having an XRPD pattern corresponding to Form 1-A. The crystalline arginine TUDCA preferably has an XRPD pattern comprising at least one, three, five, or seven peaks, in terms of 2θ, selected from the group consisting of 11.48, 15.34, 18.43, 19.19, 21.77, 23.08, and 25.29+0.2°. Alternatively, the arginine TUDCA can have an XRPD pattern substantially as depicted in FIG. 1.

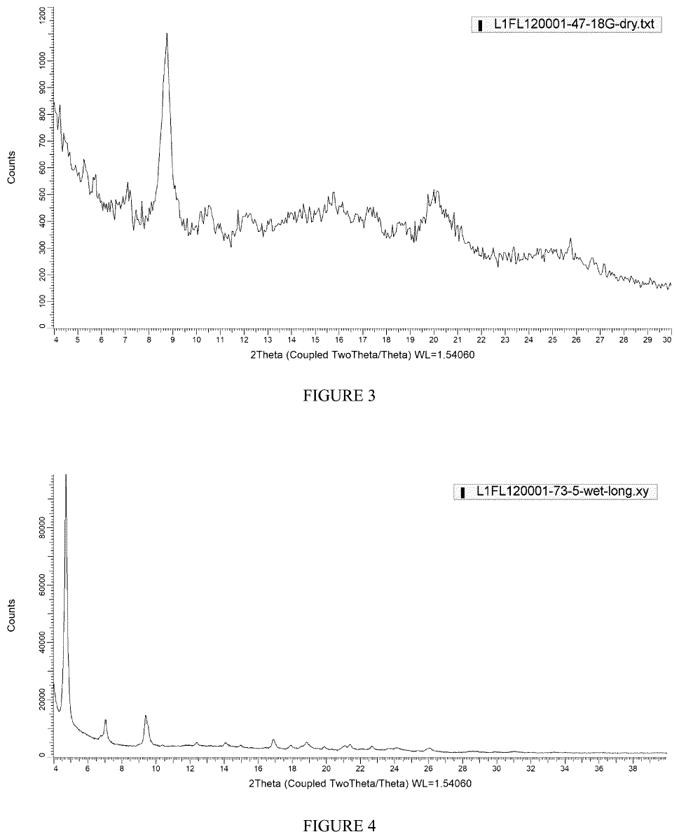

When the TUDCA is provided as lysine TUDCA, the compound will preferably be present as Form 5-A. The crystalline lysine TUDCA preferably has an XRPD pattern comprising at least one, three, or five peaks, in terms of 20, selected from the group consisting of 8.74, 10.38, 12.24, 17.25, and 20.05°±0.2°. Alternatively, the lysine TUDCA will have an XRPD pattern substantially as depicted in FIG. 3.

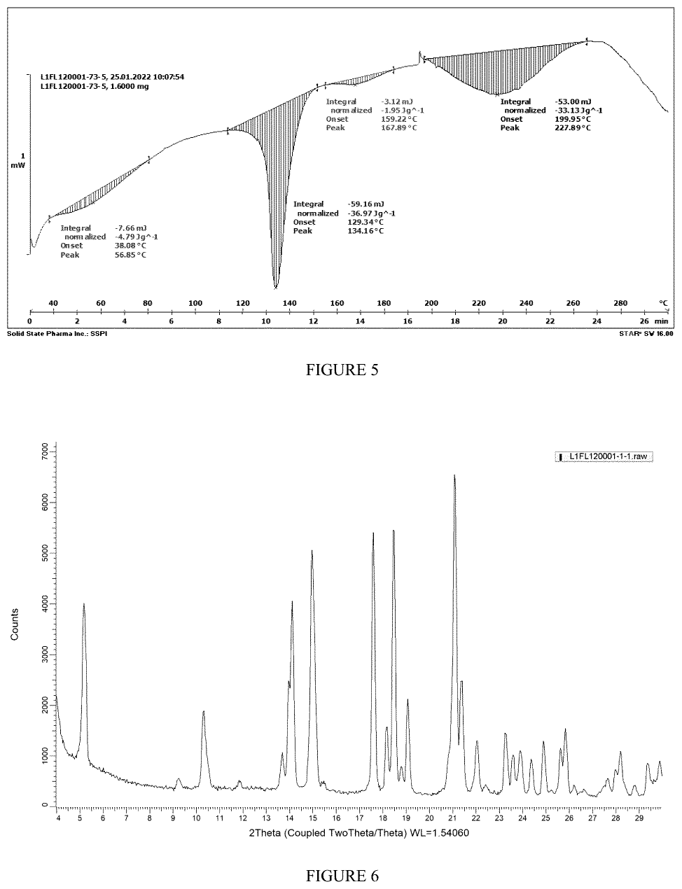

When the TUDCA is provided as histidine TUDCA, the compound will preferably be present as Form 6-A. The crystalline histidine TUDCA preferably has an XRPD pattern comprising at least one, two, or three in terms of 2θ, selected from the group consisting of 6.76, 9.40, and 12.38°±0.2°. Alternatively, the histidine TUDCA will have an XRPD pattern substantially as depicted in FIG. 5.

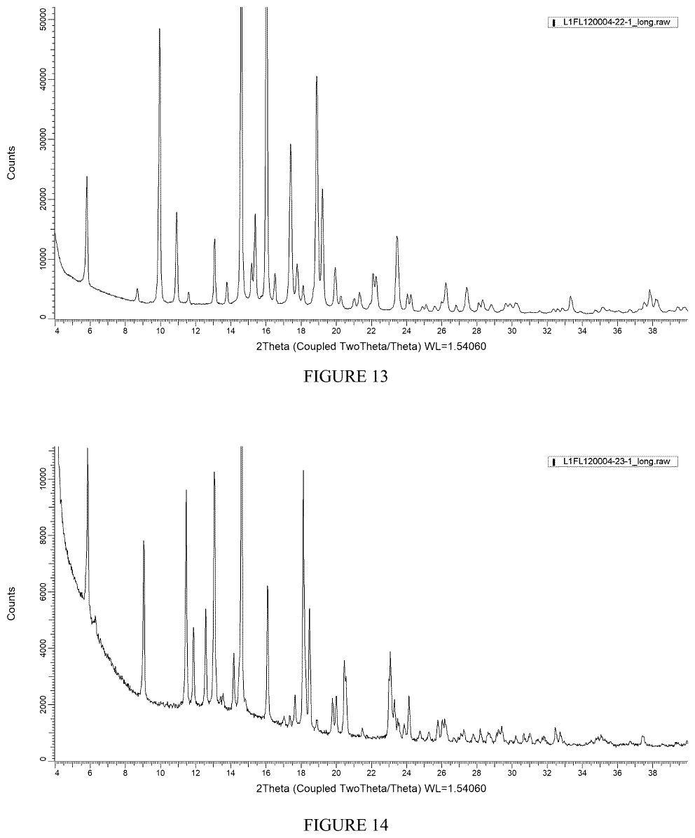

A preferred form of the ethylenediamine salt of 3,7-DKCA is a crystalline form defined by Pattern 6-D. When reference is made to a crystalline form defined by Pattern 6-D, it will be understood that the crystalline form
has an XRPD pattern comprising at least one, two, or three peaks in terms of 2θ, selected from the group consisting of 5.81, 8.69, 9.95, 10.92, 11.60, 13.08, 13.78, 14,59, 16.03, 16.51, 25.11, 27.42, 28.82, 30.24, 33.35, and 38.22° 0.2°,
has an XRPD pattern comprising at least one, two, or three peaks in terms of 2θ, selected from the group consisting of 5.81, 9.95, 10.92, 13.08, 14,69, and 16.03°±0.2°,
has an XRPD pattern comprising at least one, two, or three peaks in terms of 2θ, selected from the group consisting of 17.40, 17.77, 18.11, 18.89, 19.21, 19.94, 20.27, 210.4, 21.32, 23.45, 26.00, 26.23, 28.10, 28.33, 37.52, and 37.83° 0.1°,
has an XRPD pattern comprising at least one, two, or three peaks in terms of 2θ, selected from the group consisting of 17.40, 17.77, 18.89, 19.21, 19.94, and 23.45°±0.1°, or
has an XRPD pattern substantially as depicted in FIG. 13.

A preferred form of the tert-butylamine salt of 3,7-DKCA is a crystalline form defined by Pattern 9-A. When reference is made to a crystalline form defined by Pattern 9-A, it will be understood that the crystalline form
has an XRPD pattern comprising at least one, two, or three peaks in terms of 2θ, selected from the group consisting of 4.83, 8.77, 13.35 15.56, 16.03, 20.54, 22.05, 23.53, 24.75, 29.93, 30.40, and 31.97°±0.2°,
has an XRPD pattern comprising at least one, two, or three peaks in terms of 2θ, selected from the group consisting of 4.83, 8.77, 13.35, 15.56, and 22.05°±0.2°,
has an XRPD pattern comprising at least one, two, or three peaks in terms of 2θ, selected from the group consisting of 9.68, 9.90, 14.22, 14,46, 17.51, 17.76, 18.92, 19.30, 19.73, 20.10, 20.95, and 27.26° 2θ±0.1°,
has an XRPD pattern comprising at least one, two, or three peaks in terms of 2θ, selected from the group consisting of 9.90, 14.22, 14,46, 17.51, 19.73, and 20.10°±0.1°, or
has an XRPD pattern substantially as depicted in FIG. 12.

A preferred form of the diisopropylamine salt of 3,7-DKCA is a crystalline form defined by Pattern 10-A. When reference is made to a crystalline form defined by Pattern 10-A, it will be understood that the crystalline form
has an XRPD pattern comprising at least one, two, or three peaks in terms of 2θ, selected from the group consisting of 5.85, 6.29, 9.05, 12.58, 14.17, 16.09, 18.13, 18.47, 18.89, 20.49, 21.48, 24,75, 25.27, 28.65, 30.21, 31.82, 34.78, and 37.44°±0.2°,
has an XRPD pattern comprising at least one, two, or three peaks in terms of 2θ, selected from the group consisting of 5.85, 9.05, 12.58, 14.17, 16.09, 18.13, 18.47, and 20.49° 0.2°,
has an XRPD pattern comprising at least one, two, or three peaks in terms of 2θ, selected from the group consisting of 11.46, 11.88, 13.07, 14.61, 14.82, 17.03, 17.37, 17.65, 19.79, 20.00, 23.08, 23.86, 24.13, 25.78, 27.80, 28.19, 30.66, 31.02, 32.46, 32.74, and 35.28° 0.1°,
has an XRPD pattern comprising at least one, two, or three peaks in terms of 2θ, selected from the group consisting of 11.46, 11.88, 13.07, 14.61, 20.00, 23.08, and 24.13°±0.1°, or
has an XRPD pattern substantially as depicted in FIG. 14.

### Carbon Sources

The carbon source may be a steroid, such as cholesterol, stigmasterol, campesterol and sitosterol or mixtures of all of them, preferably sitosterol. Preferably, the carbon source will be a plant phytosterol such as sitosterol, stigmasterol, campesterol and brassicasterol or a mixture thereof. In one embodiment, the phytosterols are mainly of soybean or tall oil origin.

The origin of the carbon atoms may be even further differentiated by measurement of the δ¹³C value as disclosed e.g. in U.S. Pat. No. 8,076,156 and “Stable Isotope Ratios as biomarkers of diet for health research” by D. M. O'Brien, Annual Reviews (www.annualreviews.org), 2015. The δ-value appears as the ¹³C is measured in relation to a standard being Pee Dee Belemnite based on a Cretaceous marine fossil, which had an anomalously high ¹³C. Biochemical reactions discriminate against ¹³C, which is why the concentration of ¹²C is increased in biological materials. In this manner, different sources such as plant versus animal may be distinguished using the pure compounds as reference values as described in Application Note 30276 from Thermo Scientific: “Detection of Squalene and Squalane Origin with Flash Elemental Analyzer and Delta V Isotope Ratio Mass Spectrometer” by Guibert et al. (2013).

Isotope ratios are conveniently quantified in parts per mil (‰) in what is called the δ notation. Specifically, δ¹³C=(Rₛₐₘₚₗₑ/R_{standard}−1)×1,000 where Rₛₐₘₚₗₑ is the ¹³C/¹²C isotope ratio of the sample and R_{standard} is 0.0112372, which is based on the standard Vienna PeeDee Belemnite (VPDB) value. Thus, 1 unit of ¹³C represents a change of ˜1 in the fifth decimal place of the ¹³C/¹²C isotope ratio. Further discussion of the technique can be found, for example, in R. N. Zare et al., High-precision optical measurements of ¹³C/¹²C isotope ratios in organic compounds at natural abundance. 10928-10932, PNAS Jul. 7, 2009, vol. 106 no. 27.

The δ¹³C values may also differ among plants due to their different photosynthethic physiology. This may be observed in C3 plants such as wheat, rice, beans, most fruits and vegetables which exhibit a higher δ¹³C value than C4 plants such as corn, sugar cane and sorghum (“Stable Isotope Ratios as biomarkers of diet for health research” by D. M. O'Brien, Annual Reviews (www.annualreviews.org), 2015). In one embodiment, the TUDCA shows a δ¹³C value that is different from the δ¹³C value of TUDCA obtained from animal sources. In a further embodiment, the TUDCA shows a δ¹³C value that is different from the δ¹³C value of TUDCA obtained from mammal sources. Thus, for example, it has been experimentally determined that TUDCA made according to the present invention, in which the steroid core is derived from soy beans and the taurine from fossil sources, that the TUDCA comprises −28.95‰ δ¹³C relative to VPDB, compared to animal derived TUDCA, which can comprise as little as −14.75‰ δ¹³C relative to VPDB.

The TUDCA carbons preferably are derived predominantly from plant sources, with only a minor amount (if any) of carbons derived from non-plant sources. Thus, in various preferred embodiments the carbons in the TUDCA comprise greater than 80% plant derived carbons, with the remainder derived from non-plant sources. More particularly, the carbons in the steroidal rings are preferably 100% derived from plant sources, while any appended moieties such as taurine may be derived from non-plant sources.

### Ketoreductase Enzymes

Preferred ketoreductases have the sequences described in the examples hereto. The invention further contemplates ketoreductases having substantial identity with the sequences described in the examples, with “substantial identity” as defined herein. Thus, the invention further contemplates ketoreductases having greater than 85% identity, 90% identity, 95% identity, or 98%, to a reference sequence over a comparison window spanning 50 amino acids, 100 amino acids, 150 amino acids, 200 amino acids, 250 amino acids, or the entire amino acid sequence.

Ketoreductase enzymes having improved properties can be obtained by mutating the genetic material encoding the ketoreductase enzyme and identifying polynucleotides that express engineered enzymes with a desired property. These non-naturally occurring ketoreductases can be generated by various well-known techniques, such as in vitro mutagenesis or directed evolution. In some embodiments, directed evolution is an attractive method for generating engineered enzymes because of the relative ease of generating mutations throughout the whole of the gene coding for the polypeptide, as well as providing the ability to take previously mutated polynucleotides and subjecting them to additional cycles of mutagenesis and/or recombination to obtain further improvements in a selected enzyme property. Subjecting the whole gene to mutagenesis can reduce the bias that may result from restricting the changes to a limited region of the gene. It can also enhance generation of enzymes affected in different enzyme properties since distantly spaced parts of the enzyme may play a role in various aspects of enzyme function.

In mutagenesis and directed evolution, the parent or reference polynucleotide encoding the naturally occurring or wild type ketoreductase is subjected to mutagenic processes, for example random mutagenesis and recombination, to introduce mutations into the polynucleotide. The mutated polynucleotide is expressed and translated, thereby generating engineered ketoreductase enzymes with modifications to the polypeptide. As used herein, “modifications” include amino acid substitutions, deletions, and insertions. Any one or a combination of modifications can be introduced into the naturally occurring enzymatically active polypeptide to generate engineered enzymes, which are then screened by various methods to identify polypeptides, and corresponding polynucleotides, having a desired improvement in a specific enzyme property.

In one embodiment, the ketoreductase is not from Clostridium absonum.

### Ketoreductase Environment

The ketoreductase enzymes may be present within a cell, in the cellular medium, on an immobilized substrate, or in other forms, such as lysates and extracts of cells recombinantly designed to express the enzyme, or isolated preparations. The term “isolated polypeptide” refers to a polypeptide which is substantially separated from other contaminants that naturally accompany it, e.g., protein, lipids, and polynucleotides. The term embraces polypeptides which have been removed or purified from their naturally-occurring environment or expression system (e.g., host cell or in vitro synthesis).

In some embodiments, the isolated ketoreductase polypeptide is a substantially pure polypeptide composition. The term “substantially pure polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure ketoreductase composition will comprise about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species.

### Encoding Polynucleotide

An isolated polynucleotide encoding a ketoreductase polypeptide may be manipulated in a variety of ways to provide for expression of the polypeptide. Manipulation of the isolated polynucleotide prior to its insertion into a vector may be desirable or necessary depending on the expression vector. The techniques for modifying polynucleotides and nucleic acid sequences utilizing recombinant DNA methods are well known in the art. Guidance is provided in Sambrook et al., 2001, Molecular Cloning: A Laboratory Manual, 3^{rd} Ed., Cold Spring Harbor Laboratory Press; and Current Protocols in Molecular Biology, Ausubel. F. ed., Greene Pub. Associates, 1998, updates to 2006.

Thus, in another aspect, the present disclosure is also directed to a recombinant expression vector comprising a polynucleotide encoding a ketoreductase polypeptide or a variant thereof, and one or more expression regulating regions such as a promoter and a terminator, a replication origin, etc., depending on the type of hosts into which they are to be introduced. The various nucleic acid and control sequences may be joined together to produce a recombinant expression vector which may include one or more convenient restriction sites to allow for insertion or substitution of the nucleic acid sequence encoding the polypeptide at such sites. Alternatively, the nucleic acid sequence of the present disclosure may be expressed by inserting the nucleic acid sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus), which can be conveniently subjected to recombinant DNA procedures and can bring about the expression of the polynucleotide sequence. The choice of the vector will typically depend on the compatibility of the vector with the host cell into which the vector is to be introduced. The vectors may be linear or closed circular plasmids.

The expression vector may be an autonomously replicating vector, i.e., a vector that exists as an extrachromosomal entity, the replication of which is independent of chromosomal replication, e.g., a plasmid, an extrachromosomal element, a mini-chromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell may be used.

The term “control sequence” is defined herein to include all components, which are necessary or advantageous for the expression of a polypeptide of the present disclosure. Each control sequence may be native or foreign to the nucleic acid sequence encoding the polypeptide. Such control sequences include, but are not limited to, a leader, polyadenylation sequence, pro-peptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleic acid sequence encoding a polypeptide.

The term “operably linked” is defined herein is a configuration in which a control sequence is appropriately placed at a position relative to the coding sequence of the DNA sequence such that the control sequence directs the expression of a polynucleotide and/or polypeptide. The control sequence may be an appropriate promoter sequence. The “promoter sequence” is a nucleic acid sequence that is recognized by a host cell for expression of the coding region. The promoter sequence contains transcriptional control sequences, which mediate the expression of the polypeptide. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell. The control sequence may also be a suitable transcription terminator sequence, a sequence recognized by a host cell to terminate transcription. The terminator sequence is operably linked to the 3′ terminus of the nucleic acid sequence encoding the polypeptide. Any terminator which is functional in the host cell of choice may be used in the present invention.

### Host Cells for Expression of Ketoreductase Polypeptides

In another aspect, the present disclosure provides a host cell comprising a polynucleotide encoding a ketoreductase polypeptide of the present disclosure, the polynucleotide being operatively linked to one or more control sequences for expression of the ketoreductase enzyme in the host cell. Host cells for use in expressing the KRED polypeptides encoded by the expression vectors of the present invention are well known in the art and include but are not limited to, bacterial cells, such as E. coli cells; fungal cells, such as yeast cells (e.g., Saccharomyces cerevisiae or Pichia pastoris). In one particular embodiment, the process of the current invention is carried out with whole cells that express the 3-ketoreductase, or an extract or lysate of such cells, wherein the whole cells or extract or lysate of such whole cells are selected from Escherichia coli, Pichia pastoris or Saccharomyces cerevisiae. Appropriate culture mediums and growth conditions for the above-described host cells are well known in the art.

Polynucleotides for expression of the ketoreductase may be introduced into cells by various methods known in the art. For the bacterial systems and yeasts described herein, the typical process is by transformation (e.g. electroporation or calcium chloride mediated) or conjugation, or sometimes protoplast fusion. Various methods for introducing polynucleotides into cells will be apparent to the skilled artisan.

### Cofactors

As is known by those of skill in the art, ketoreductase-catalyzed reduction reactions typically require a cofactor. As used herein, the term “cofactor” refers to a non-protein compound that operates in combination with a ketoreductase enzyme. Cofactors suitable for use with the ketoreductase enzymes described herein include, but are not limited to, NADP⁺ (nicotinamide adenine dinucleotide phosphate), NADPH (the reduced form of NADP⁺), NAD⁺ (nicotinamide adenine dinucleotide) and NADH (the reduced form of NAD⁺). The weight ratio of the cofactor to the 3-ketoreductase is commonly from about 10:1 to 100:1.

The following equation illustrates an embodiment of a ketoreductase catalyzed reduction reaction utilizing NADH or NADPH as a cofactor, which are represented as alternatives by the designation NAD(P)H:

3-keto-sterol+NAD(P)H+H⁺+KRED→3-beta-hydroxy-sterol+NAD(P)⁺

The reduced NAD(P)H form can be optionally regenerated from the oxidized NAD(P)⁺ form using a cofactor regeneration system. The term “cofactor regeneration system” refers to a set of reactants that participate in a reaction that reduces the oxidized form of the cofactor (e.g., NADP⁺ to NADPH). Cofactors oxidized by the ketoreductase-catalyzed reduction of the 3-keto-sterol are regenerated in reduced form by the cofactor regeneration system. Cofactor regeneration systems comprise a stoichiometric reductant that is a source of reducing hydrogen equivalents and is capable of reducing the oxidized form of the cofactor. The cofactor regeneration system may further comprise a catalyst, for example an enzyme catalyst, that catalyzes the reduction of the oxidized form of the cofactor by the reductant.

Exemplary cofactor regeneration systems that may be employed include, but are not limited to, glucose and glucose dehydrogenase, formate and formate dehydrogenase, glucose-6-phosphate and glucose-6-phosphate dehydrogenase, a secondary (e.g., isopropanol) alcohol and secondary alcohol dehydrogenase, phosphite and phosphite dehydrogenase, molecular hydrogen and dehydrogenase, and the like. These systems may be used in combination with either NADP⁺/NADPH or NAD⁺/NADH as the cofactor.

In some embodiments, when the process is carried out using whole cells of the host organism, the whole cell may natively provide the cofactor. Alternatively or in combination, the cell may natively or recombinantly provide the cofactor.

### Reaction Conditions

In carrying out the stereoselective reductions described herein, the ketoreductase enzyme, and any enzymes comprising the optional cofactor regeneration system, may be added to the reaction mixture in the form of the purified enzymes (including immobilized variants), whole cells transformed with gene(s) encoding the enzymes, and/or cell extracts and/or lysates of such cells.

The gene(s) encoding the engineered ketoreductase enzyme and the optional cofactor regeneration enzymes can be transformed into host cells separately or together into the same host cell.

For example, in some embodiments one set of host cells can be transformed with gene(s) encoding the ketoreductase enzyme and another set can be transformed with gene(s) encoding the cofactor regeneration enzymes. Both sets of transformed cells can be utilized together in the reaction mixture in the form of whole cells, or in the form of lysates or extracts derived therefrom. In other embodiments, a host cell can be transformed with gene(s) encoding both the engineered ketoreductase enzyme and the cofactor regeneration enzymes.

Whole cells transformed with gene(s) encoding the ketoreductase enzyme and/or the optional cofactor regeneration enzymes, or cell extracts and/or lysates thereof, may be employed in a variety of different forms, including solid (e.g., lyophilized, spray-dried, immobilized, and the like) or semisolid (e.g., a crude paste). The cell extracts or cell lysates may be partially purified by precipitation (ammonium sulfate, polyethyleneimine, heat treatment or the like), followed by a desalting procedure prior to lyophilization (e.g., ultrafiltration, dialysis, and the like).

The quantities of reactants used in the reduction reaction will generally vary depending on the quantities of ketoreductase substrate employed. The following guidelines can be used to determine the amounts of ketoreductase, cofactor, and optional cofactor regeneration system to use. Generally, 3-keto-sterol substrates are employed at a concentration of about 20 to 300 grams/liter using from about 50 mg/liter to about 5 g/liter of ketoreductase and about 10 mg/liter to about 150 mg/liter of cofactor. The weight ratio of Compound 1 or Compound 2 to the 3-ketoreductase in the reaction mixture is commonly from about 10:1 to 200:1. Those having ordinary skill in the art will readily understand how to vary these quantities to tailor them to the desired level of productivity and scale of production.

Appropriate quantities of optional cofactor regeneration system may be readily determined by routine experimentation based on the amount of cofactor and/or ketoreductase utilized. In general, the reductant (e.g., glucose, formate, isopropanol) is utilized at levels above the equimolar level of ketoreductase substrate to achieve essentially complete or near complete conversion of the ketoreductase substrate.

The order of addition of reactants is not critical. The reactants may be added together at the same time to a solvent (e.g., monophasic solvent, biphasic aqueous co-solvent system, and the like), or alternatively, some of the reactants may be added separately, and some together at different time points. For example, the cofactor regeneration system, cofactor, ketoreductase, and ketoreductase substrate may be added first to the solvent. Preferably, however, the enzyme preparation is added last.

Suitable conditions for carrying out the ketoreductase-catalyzed reduction reactions described herein include a wide variety of conditions including contacting the ketoreductase enzyme and substrate at an experimental pH and temperature and detecting product, for example, using the methods described in the Examples provided herein.

The ketoreductase-catalyzed reduction reactions described herein are generally carried out in a solvent. Suitable solvents include water, organic solvents (e.g., ethyl acetate, butyl acetate, 1-octanol, heptane, octane, methyl t-butyl ether (MTBE), toluene, and the like), ionic liquids (e.g., 1-ethyl 4-methylimidazolium tetrafluoroborate, 1-butyl-3-methylimidazolium tetrafluoroborate, 1-butyl-3-methylimidazolium hexafluorophosphate, and the like). In some embodiments, aqueous solvents, including water and aqueous co-solvent systems, are used. The solvent system is preferably greater than 50%, 75%, 90%, 95%, or 98% water, and in one embodiment is 100% water.

During the course of the reduction reactions, the pH of the reaction mixture may change. The pH of the reaction mixture may be maintained at a desired pH or within a desired pH range by the addition of an acid or a base during the course of the reaction. Alternatively, the pH may be controlled by using a solvent that comprises a buffer. Suitable buffers to maintain desired pH ranges are known in the art and include, for example, phosphate buffer, triethanolamine buffer, and the like. Combinations of buffering and acid or base addition may also be used.

The ketoreductase catalyzed reduction is typically carried out at a temperature in the range of from about 15° C. to about 75° C. For some embodiments, the reaction is carried out at a temperature in the range of from about 20° C. to about 55° C. In still other embodiments, it is carried out at a temperature in the range of from about 20° C. to about 45° C. The reaction may also be carried out under ambient conditions.

The reduction reaction is generally allowed to proceed until essentially complete, or near complete, reduction of substrate is obtained. Reduction of substrate to product can be monitored using known methods by detecting substrate and/or product. Suitable methods include gas chromatography, HPLC, TLC and the like. Conversion yields of the sterol reduction product generated in the reaction mixture are generally greater than about 50%, may also be greater than about 60%, may also be greater than about 70%, may also be greater than about 80%, may also be greater than 90%, and can even be greater than about 97% or 99%.

The keto-reduction product can be recovered from the reaction mixture and optionally further purified using methods that are known to those of skill in the art. Chromatographic techniques for isolation of the keto-reduction product include, among others, reverse-phase and normal-phase chromatography. A preferred method for product purification involves extraction into an organic solvent and subsequent crystallization.

### Dosage Forms Routes of Administration

Pharmaceutical compositions (which by definition includes dietary supplements and other manufactured dosage forms) for preventing and/or treating a subject are further provided comprising a therapeutically effective amount of TUDCA, or a salt thereof, and one or more pharmaceutically acceptable excipients. A “pharmaceutically acceptable excipient” is one that is not biologically or otherwise undesirable, i.e., the material can be administered to a subject without causing any undesirable biological effects or interacting in a deleterious manner with any of the other components of the pharmaceutical composition in which it is contained. The carrier can be selected to minimize any degradation of the active ingredient and to minimize any adverse side effects in the subject, as would be well known to one of skill in the art. The carrier can be a solid, a liquid, or both.

The disclosed compounds can be administered by any suitable route, preferably in the form of a pharmaceutical composition adapted to such a route, and in a dose effective for the treatment or prevention intended. In a preferred embodiment, the active compounds and compositions, are administered orally. Suitable carriers and their formulations are described in Remington: The Science and Practice of Pharmacy (19th ed.) ed. A. R. Gennaro, Mack Publishing Company, Easton, Pa., 1995. Oral administration of a solid dose form can be, for example, presented in discrete units, such as hard or soft capsules, pills, sachets, lozenges, or tablets, each containing a predetermined amount of at least one of the disclosed compound or compositions. In some forms, the oral administration can be in a powder or granule form. In the case of capsules, tablets, and pills, the dosage forms also can comprise buffering agents or can be prepared with enteric coatings.

Preferred embodiments are described by Embodiments 1-53 below:

[Embodiment 1] A compound selected from a taurine conjugate of ursodeoxycholic acid of formula I (TUDCA):

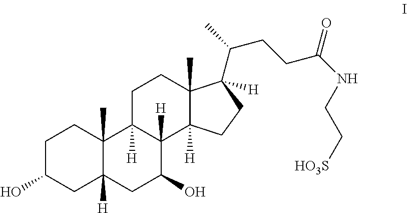

and its salts comprising a δ¹³C value corresponding to a plant derived molecule, preferably comprising less than −20‰, −22.5‰, or −25‰ 613C relative to VPDB.

[Embodiment 2] A compound selected from a taurine conjugate of ursodeoxycholic acid of formula I (TUDCA):

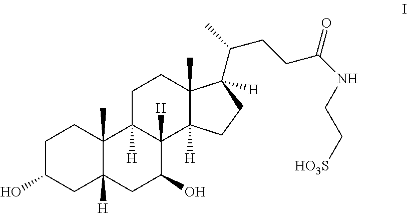

and its salts comprising an impurity profile characterized by: (a) less than 0.1%, 0.05%, 0.03%, or 0.01% of UDCA; (b) less than 0.1%, 0.05%, 0.03%, or 0.01% of taurine; (c) less than 1.0%. 0.50%, 0.30% or 0.10% of 5α-TUDCA, optionally greater than 0.005% of 5α-TUDCA; (d) less than 0.20%, 0.10%, 0.05% of TCDCA; and/or (e) a combination thereof.

[Embodiment 3] A crystalline plant-derived taurine conjugate of ursodeoxycholic acid of formula I (TUDCA):

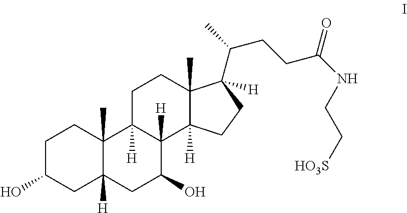

comprising: (a) an XRPD pattern corresponding to Form A TUDCA or Form L TUDCA; and (b) a δ¹³C value corresponding to a plant derived molecule.

[Embodiment 4] A crystalline taurine conjugate of ursodeoxycholic acid of formula I (TUDCA):

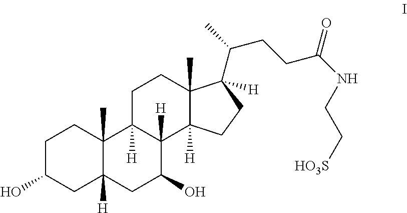

comprising an XRPD pattern corresponding to Form L TUDCA.

[Embodiment 5] A salt of a taurine conjugate of ursodeoxycholic acid of formula I selected from the group consisting of arginine TUDCA, histidine TUDCA, and lysine TUDCA:

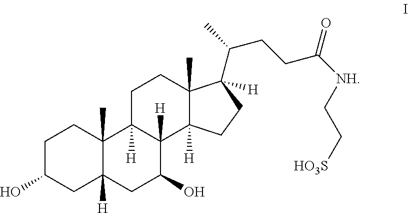

[Embodiment 6] The compound of Embodiment 1 or 2, having an XRPD pattern corresponding to Form A TUDCA or Form L TUDCA.

[Embodiment 7] The compound of Embodiment 1 or 2 (Form A), having an X-ray powder diffraction pattern comprising at least one, three, or five peaks, in terms of 2θ, selected from the group consisting of 5.19, 10.31, 10.49, 19.08, 20.83, 22.03, 23.26, 23.58, 24.89, and 31.09±0.2°, preferably 1, 2, or 3 peaks, in terms of 2θ, selected from the group consisting of 5.19, 10.31, 19.08, and 31.09±0.2°.

[Embodiment 8] The compound of Embodiment 1 or 2 (Form A), having an X-ray powder diffraction pattern substantially as depicted in FIG. 6.

[Embodiment 9] The compound of Embodiment 1 or 2 (Form L), having an X-ray powder diffraction pattern comprising at one or two peaks, in terms of 2θ, selected from the group consisting of 4.59 and 19.610±0.2°, optionally in combination with one or any combination of 15.11, 17.56, 18.41, and 21.38° 0.2°.

[Embodiment 10] The compound of Embodiment 1 or 2 (Form L), having an X-ray powder diffraction pattern substantially as depicted in FIG. 8.

[Embodiment 11] The arginine TUDCA of Embodiment 1, 2, or 5, having an XRPD pattern corresponding to Form 1-A.

[Embodiment 12] The arginine TUDCA of Embodiment 1, 2, or 5 (Form 1-A), having an XRPD pattern comprising at least one, three, five, or seven peaks, in terms of 2θ, selected from the group consisting of 11.48, 15.34, 18.43, 19.19, 21.77, 23.08, and 25.29±0.2°.

[Embodiment 13] The arginine TUDCA of Embodiment 1, 2, or 5 (Form 1-A), having an XRPD pattern substantially as depicted in FIG. 1.

[Embodiment 14] The lysine TUDCA of Embodiment 1, 2, or 5 (Form 5-A), having an XRPD pattern comprising at least one, three, or five peaks, in terms of 2θ, selected from the group consisting of 8.74, 10.38, 12.24, 17.25, and 20.050±0.2°.

[Embodiment 15] The lysine TUDCA of Embodiment 1, 2, or 5 (Form 5-A), having an XRPD pattern substantially as depicted in FIG. 3.

[Embodiment 16] The histidine TUDCA of Embodiment 1, 2, or 5 (Form 6-A), having an XRPD pattern comprising at least one, two, or three in terms of 2θ, selected from the group consisting of 6.76, 9.40, and 12.38°±0.2°.

[Embodiment 17] The histidine TUDCA of Embodiment 1, 2, or 5 (Form 6-A), having an XRPD pattern substantially as depicted in FIG. 5.

[Embodiment 18] The compound of Embodiment 1, 3, 4, or 5, comprising an impurity profile characterized by: (a) less than 5%, 3%, 1%, 0.5%, 0.1%, 0.05%, 0.03%, or 0.01% of UDCA; (b) less than 5%, 3%, 1%, 0.5%, 0.1%, 0.05%, 0.03%, or 0.01% of taurine; (c) less than 5%, 3%, 1%, 0.5%, 0.1%, 0.05%, 0.03%, or 0.01% of any 3 β-hydroxysteroids; (d) less than 5%, 3%, 1%, 0.5%, 0.1%, 0.05%, 0.03%, or 0.01% of any 5α-steroids, optionally greater than 0.005% of any 5α-steroids; or (e) less than 5%, 3%, 1%, 0.5%, 0.1%, 0.05%, 0.03%, or 0.01% of any 7α-hydroxysteroids.

[Embodiment 19] The compound of Embodiment 1, 2, 3, 4, or 5, made by a process that goes through a TDKCA intermediate, comprising: (a) contacting the TDKCA with a 3α-hydroxysteroid dehydrogenase to stereo-selectively reduce the TDKCA to a 3α-hydroxy intermediate, and contacting the 3α-hydroxy intermediate with a 7β-hydroxysteroid dehydrogenase to stereo-selectively reduce the 3α-hydroxy intermediate to TUDCA; (b) contacting the TDKCA with a 7β-hydroxysteroid dehydrogenase to stereo-selectively reduce the TDKCA to a 7β-hydroxy intermediate, and contacting the 7β-hydroxy intermediate with a 3α-hydroxysteroid dehydrogenase to stereo-selectively reduce the 7β-hydroxy intermediate to TUDCA; or (c) simultaneously contacting the TDKCA with a 3α-hydroxysteroid dehydrogenase and a 7β-hydroxysteroid dehydrogenase to stereo-selectively reduce the TDKCA to TUDCA.

[Embodiment 20] The compound of Embodiment 1, 3, 4, or 5, comprising an impurity profile characterized by: (a) less than 1% of UDCA; (b) less than 1% of taurine; (c) less than 1% of any 3β-hydroxysteroids; (d) less than 1% of any 5α-steroids, optionally greater than 0.005% of any 5α-steroids; and/or (e) less than 1% of any 7α-hydroxysteroids.

[Embodiment 21] The compound of Embodiment 1, 3, 4, or 5, comprising an impurity profile characterized by: (a) less than 0.1% of UDCA; (b) less than 0.1% of taurine; (c) less than 0.1% of any 3β-hydroxysteroids; (d) less than 0.1% of any 5α-steroids, optionally greater than 0.005% of any 5α-steroids; and/or(e) less than 0.1% of any 7α-hydroxysteroids.

[Embodiment 22] The compound of Embodiment 1, 3, 4, or 5, comprising an impurity profile characterized by: (a) less than 5%, 3%, 1%, 0.5%, 0.1%, 0.05%, 0.03%, or 0.01% of UDCA; (b) less than 5%, 3%, 1%, 0.5%, 0.1%, 0.05%, 0.03%, or 0.01% of taurine; (c) less than 5%, 3%, 1%, 0.5%, 0.1%, 0.05%, 0.03%, or 0.01% of any 3 β-hydroxysteroids; (d) less than 5%, 3%, 1%, 0.5%, 0.1%, 0.05%, 0.03%, or 0.01% of any 5α-steroids optionally greater than 0.005% of any 5α-steroids; and (e) less than 5%, 3%, 1%, 0.5%, 0.1%, 0.05%, 0.03%, or 0.01% of any 7α-hydroxysteroids.

[Embodiment 23] The compound of Embodiment 1, 3, 4, or 5, comprising an impurity profile characterized by: (a) less than 1% of UDCA; (b) less than 1% of taurine; (c) less than 1% of any 3β-hydroxysteroids; (d) less than 1% of any 5α-steroids, optionally greater than 0.005% of any 5α-steroids; and (e) less than 1% of any 7α-hydroxysteroids.

[Embodiment 24] The compound of Embodiment 1, 2, 3, 4, or 5, comprising an impurity profile characterized by: (a) less than 0.1% of UDCA; (b) less than 0.1% of taurine; (c) less than 0.1% of any 3β-hydroxysteroids; (d) less than 0.1% of any 5α-steroids, optionally greater than 0.005% of any 5α-steroids; and (e) less than 0.1% of any 7α-hydroxysteroids.

[Embodiment 25] The compound of Embodiment 1, 2, 3, 4, or 5, comprising: (a) less than 5%, 3%, 1%, 0.5%, 0.1%, 0.05%, 0.03%, or 0.01% of any 5α-steroids, optionally greater than 0.005% of any 5α-steroids; (b) less than 5%, 3%, 1%, 0.5%, 0.1%, 0.05%, 0.03%, or 0.01% of any 3-keto, 7-hydroxysteroids; (c) less than 5%, 3%, 1%, 0.5%, 0.1%, 0.05%, 0.03%, or 0.01% of any 3-hydroxy, 7-ketosteroids; and/or (d) less than 5%, 3%, 1%, 0.5%, 0.1%, 0.05%, 0.03%, or 0.01% of TDKCA.

[Embodiment 26] The compound of Embodiment 1, 2, 3, 4, or 5, comprising: (a) less than 5%, 3%, 1%, 0.5%, 0.1%, 0.05%, 0.03%, or 0.01% of any 5α-steroids; optionally greater than 0.005% of any 5α-steroids; (b) less than 5%, 3%, 1%, 0.5%, 0.1%, 0.05%, 0.03%, or 0.01% of any 3-keto, 7-hydroxysteroids; (c) less than 5%, 3%, 1%, 0.5%, 0.1%, 0.05%, 0.03%, or 0.01% of any 3-hydroxy, 7-ketosteroids; (d) less than 5%, 3%, 1%, 0.5%, 0.1%, 0.05%, 0.03%, or 0.01% of TLCA; (e) less than 5%, 3%, 1%, 0.5%, 0.1%, 0.05%, 0.03%, or 0.01% of TDKCA; and/or (f) less than 5%, 3%, 1%, 0.5%, 0.1%, 0.05%, 0.03%, or 0.01% of any 3β-hydroxysteroids.

[Embodiment 27] The compound of any of Embodiments 2, 4, or 5, comprising a δ¹³C value corresponding to a plant derived molecule or a mixed fossil and plant derived molecule, preferably comprising less than −20‰, −22.5‰, or −25‰ 613C relative to VPDB.

[Embodiment 28] The compound of any of Embodiments 1-27 in an isolated state.

[Embodiment 29] The compound of any of Embodiments 1-28 comprising less than 3%, 2%, or 1% impurities selected from starting materials, by-products, intermediates, and degradation products.

[Embodiment 30] The compound of any of Embodiments 1-28 comprising less than 1% or 0.5% of impurities selected from starting materials, by-products, intermediates, and degradation products.

[Embodiment 31] A pharmaceutical composition comprising the compound of any of Embodiments 1-30 and one or more pharmaceutically acceptable excipients.

[Embodiment 32] A method of making a TUDCA pharmaceutical dosage form comprising admixing the compound of any of Embodiments 1-30 with one or more pharmaceutically acceptable excipients to form an admixture and processing the admixture into a finished dosage form, optionally by compressing the admixture into a tablet or filling the admixture into a capsule or sachet.

[Embodiment 33] A method of producing the compound of any of Embodiments 1-30 that goes through a TDKCA intermediate comprising: (a) contacting the TDKCA with a 3α-hydroxysteroid dehydrogenase to stereo-selectively reduce the TDKCA to a 3α-hydroxy intermediate, and contacting the 3α-hydroxy intermediate with a 7β-hydroxysteroid dehydrogenase to stereo-selectively reduce the 3α-hydroxy intermediate to TUDCA; (b) contacting the TDKCA with a 7β-hydroxysteroid dehydrogenase to stereo-selectively reduce the TDKCA to a 7β-hydroxy intermediate, and contacting the 7β-hydroxy intermediate with a 3α-hydroxysteroid dehydrogenase to stereo-selectively reduce the 7β-hydroxy intermediate to TUDCA; or (c) simultaneously contacting the TDKCA with a 3α-hydroxysteroid dehydrogenase and a 7β-hydroxysteroid dehydrogenase to stereo-selectively reduce the TDKCA to TUDCA.

[Embodiment 34] The method of Embodiment 33, carried out with whole cells that express the 3α-hydroxysteroid dehydrogenase, the 7β-hydroxysteroid dehydrogenase, or both, or an extract or lysate of such cells, wherein the whole cells or extract or lysate of such whole cells are selected from native or recombinant bacteria or yeast, preferably Escherichia coli, Pichia pastoris or Saccharomyces cerevisiae.

[Embodiment 35] The method of Embodiment 33 or 34, wherein the TDKCA is derived from: (a) an ethylenediamine salt of 3,7-DKCA, optionally a crystalline form defined by Pattern 6-D, (b) a tert-butylamine salt of 3,7-DKCA, optionally a crystalline form defined by Pattern 9-A, or (c) a diisopropylamine salt of 3,7-DKCA, optionally a crystalline form defined by Pattern 10-A.

[Embodiment 36] The method of Embodiment 33 or 34, wherein the TDKCA is made by: (a) providing a precursor compound selected from an ethylenediamine salt of 3,7-DKCA, optionally a crystalline form defined by Pattern 6-D, a tert-butylamine salt of 3,7-DKCA, optionally a crystalline form defined by Pattern 9-A, a diisopropylamine salt of 3,7-DKCA, optionally a crystalline form defined by Pattern 10-A, or an ester thereof; (b) optionally, when starting with either the ethylenediamine salt of 3,7-DKCA, the tert-butylamine salt of 3,7-DKCA, or the diisopropylamine salt of 3,7-DKCA, converting the salt to a free acid; (c) contacting the 24-carboxylic acid or ester group with a reagent that converts the acid or ester group to a derivative that can act as an acylating agent; and (d) reacting the derivative with taurine to form TDKCA or a salt thereof.

[Embodiment 37] A method of making TUDCA or a salt thereof comprising: (a) (i) contacting 3,7-DKCA with a 3 α-hydroxysteroid dehydrogenase to stereo-selectively reduce the 3,7-KDCA to a 3α-hydroxy intermediate, and contacting the 3α-hydroxy intermediate with a 7β-hydroxysteroid dehydrogenase to stereo-selectively reduce the 3α-hydroxy intermediate to UDCA; or (ii) contacting the 3,7-DKCA with a 7β-hydroxysteroid dehydrogenase to stereo-selectively reduce the 3,7-DKCA to a 7β-hydroxy intermediate, and contacting the 7β-hydroxy intermediate with a 3 α-hydroxysteroid dehydrogenase to stereo-selectively reduce the 7β-hydroxy intermediate to UDCA; or (iii) simultaneously contacting the 3,7-DKCA with a 3α-hydroxysteroid dehydrogenase and a 7β-hydroxysteroid dehydrogenase to stereo-selectively reduce the 3,7-DKCA to UDCA, and (b) conjugating the UDCA with taurine to form TUDCA, wherein the 3,7-DKCA is provided as or derived from an ethylenediamine salt of 3,7-DKCA (optionally Pattern 6-D), a tert-butylamine salt of 3,7-DKCA (optionally Pattern 9-A), or a diisopropylamine salt of 3,7-DKCA (optionally Pattern 10-A).

[Embodiment 38] The method of Embodiment 37, wherein step (b) is performed by: (a) contacting the 24-carboxylic acid of UDCA with a reagent that converts the acid group to a derivative that can act as an acylating agent; and (b) reacting the derivative with taurine to form TDKCA or a salt thereof.

[Embodiment 39] The method of any of Embodiments 33-38, further comprising isolating the TUDCA.

[Embodiment 40] The method of any of Embodiments 33-39, further comprising admixing the TUDCA with one or more pharmaceutically acceptable excipients to form an admixture and processing the admixture into a finished dosage form, optionally by compressing the admixture into a tablet or filling the admixture into a capsule or sachet.

[Embodiment 41]3α-Hydroxy-7-oxo-5β-cholanoyltaurine or a salt thereof having the following chemical structure:

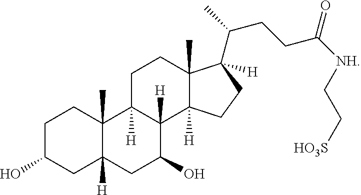

[Embodiment 42] The 3α-Hydroxy-7-oxo-5β-cholanoyltaurine of Embodiment 41 in its free form, optionally in the substantial absence of any salt forms.

[Embodiment 43]70-Hydroxy-3-oxo-5β-cholanoyltaurine or a salt thereof having the following chemical structure:

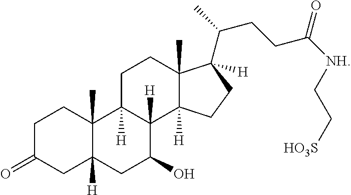

[Embodiment 44] The 7β-Hydroxy-3-oxo-5β-cholanoyltaurine of Embodiment 43 in its free form, optionally in the substantial absence of any salt forms.

[Embodiment 45]3,7-Oxo-5β-cholanoyltaurine or a salt thereof having the following chemical structure:

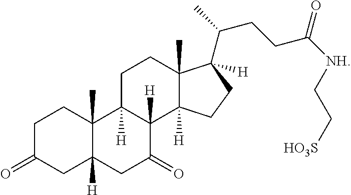

[Embodiment 46] The 3,7-Oxo-5β-cholanoyltaurine of Embodiment 45 in its free form, optionally in the substantial absence of any salt forms.

[Embodiment 47] An ethylenediamine salt of 3,7-DKCA.

[Embodiment 48] The ethylenediamine salt of 3,7-DKCA of Embodiment 47 having crystalline form Pattern 6-D defined by: (a) an XRPD pattern comprising at least one, two, or three peaks in terms of 2θ, selected from the group consisting of 5.81, 8.69, 9.95, 10.92, 11.60, 13.08, 13.78, 14,59, 16.03, 16.51, 25.11, 27.42, 28.82, 30.24, 33.35, and 38.220±0.2°, or (b) an XRPD pattern substantially as depicted in FIG. 13.

[Embodiment 49] A tert-butylamine salt of 3,7-DKCA.

[Embodiment 50] The tert-butylamine salt of 3,7-DKCA of Embodiment 49 having crystalline form Pattern 9-A defined by: (a) an XRPD pattern comprising at least one, two, or three peaks in terms of 2θ, selected from the group consisting of 4.83, 8.77, 13.35 15.56, 16.03, 20.54, 22.05, 23.53, 24.75, 29.93, 30.40, and 31.97°±0.2°, or (b) an XRPD pattern substantially as depicted in FIG. 12.

[Embodiment 51] A diisopropylamine salt of 3,7-DKCA.

[Embodiment 52] The diisopropylamine salt of 3,7-DKCA of Embodiment 51 having crystalline form Pattern 10-A defined by (a) an XRPD pattern comprising at least one, two, or three peaks in terms of 2θ, selected from the group consisting of 5.85, 6.29, 9.05, 12.58, 14.17, 16.09, 18.13, 18.47, 18.89, 20.49, 21.48, 24,75, 25.27, 28.65, 30.21, 31.82, 34.78, and 37.440+0.2°, or (b) has an XRPD pattern substantially as depicted in FIG. 14.

[Embodiment 53] The compound of any of Embodiments 41-52 in an isolated state.

[Embodiment 54] TUDCA or a salt thereof comprising ≤0.1% of any 3-3 impurities, ≤0.1% of any 5-α impurities, and ≤0.1% of any 7-α impurities.

[Embodiment 55] TUDCA or a salt thereof comprising ≤0.05% of any 3-3 impurities, ≤0.05% of any 5-α impurities, and ≤0.05% of any 7-α impurities.

[Embodiment 56] The TUDCA of Embodiment 54 or 55, or salt thereof, comprising ≤0.1% UDCA and ≤0.1% taurine.

[Embodiment 57] The TUDCA of Embodiment 54 or 55, or salt thereof, comprising ≤0.05% UDCA and ≤0.05% taurine.

[Embodiment 58] TUDCA comprising less than −20‰ 613C relative to VPDB, <0.1% of any 3-3 impurities, ≤0.1% of any 5-α impurities, and ≤0.1% of any 7-α impurities.

[Embodiment 59] TUDCA comprising less than −20‰ 613C relative to VPDB, ≤0.05% of any 3-3 impurities, K 0.05% of any 5-α impurities, and ≤0.05% of any 7-α impurities.

[Embodiment 60] The TUDCA of Embodiment 58 or 59, or salt thereof, comprising ≤0.1% UDCA and ≤0.1% taurine.

[Embodiment 61] The TUDCA of Embodiment 58 or 59, or salt thereof, comprising ≤0.05% UDCA and ≤0.05% taurine.

[Embodiment 62] The TUDCA of Embodiment 58, 59, 60, or 61, comprising less than 22.5‰ 613C relative to VPDB.

[Embodiment 63] The TUDCA of Embodiment 58, 59, 60, or 61, comprising less than 25‰ 613C relative to VPDB.

[Embodiment 64] The TUDCA of Embodiment 54, 55, 56, 57, 58, 59, 60, 61, 62, or 63, in an isolated state.

[Embodiment 65] The TUDCA of Embodiment 54, 55, 56, 57, 58, 59, 60, 61, 62, or 63, in a pharmaceutical dosage form comprising one or more pharmaceutically acceptable excipients.

[Embodiment 66] The TUDCA of Embodiment 54, 55, 56, 57, 58, 59, 60, 61, 62, or 63, in a powder sachet comprising one or more pharmaceutically acceptable excipients.

### EXAMPLES

In the following examples, efforts have been made to ensure accuracy with respect to numbers (e.g., amounts, temperature, etc.) but some errors and deviations should be accounted for.

The following examples are put forth so as to provide those of ordinary skill in the art with a complete disclosure and description of how the methods claimed herein are made and evaluated and are intended to be purely exemplary of the invention and are not intended to limit the scope of what the inventors regard as their invention.

### Example 1. Preparation of KCEA from Bisnoralcohol

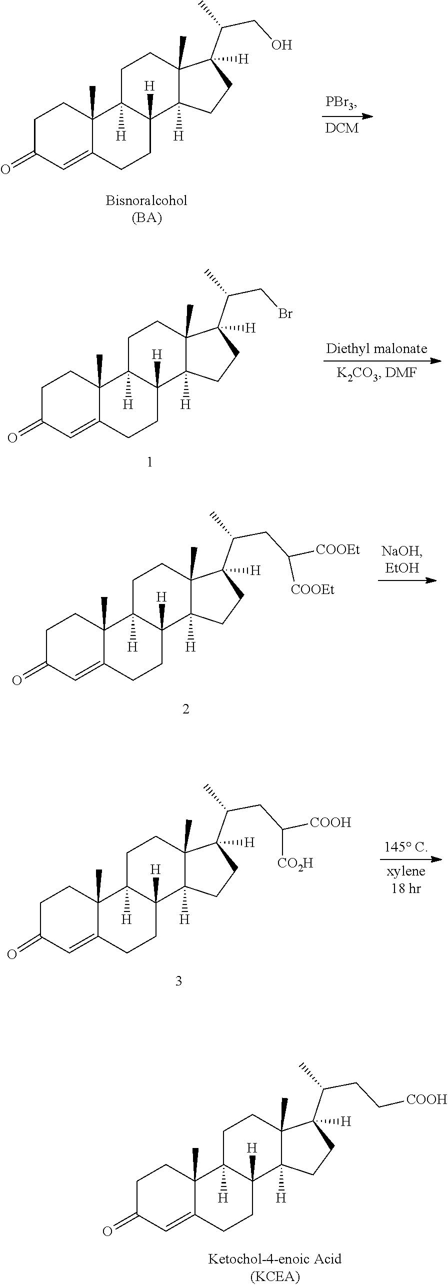

### Bromination of Bisnoralcohol:

To a stirred solution of bisnoralcohol (BA, 1 g, 3.02 mmol) in dichloromethane (DCM, 20 mL) was added PBr₃ (0.34 mL, 3.63 mmol) at 0° C. The mixture was warmed to room temperature and stirred for 3 hr, at which point TLC analysis showed complete conversion of starting material. The reaction mixture was quenched using ice water (10 mL), stirred for 15 min and the layers were separated. The aqueous layer was extracted in DCM (10 mL) and the combined organic phase was concentrated under reduced pressure to afford compound 1 as a yellow gummy oil (crude yield 1.2 g). ¹H NMR (400 MHz, DMSO-d6): δ 5.61 (s, 1H), 3.56-3.51 (m, 1H), 3.45 (dd, J=2.1 Hz and 1.1 Hz, 1H), 2.46-2.32 (m, 2H), 2.26-2.10 (m, 2H), 199-1.89 (m, 3H), 1.82-1.70 (m, 2H), 1.70-0.82 (m, 18H), 0.70 (s, 3H) ppm.

Alkylation of Diethyl Malonate with Compound 1:

To a stirred solution of compound 1 (0.5 g, 1.27 mmol) in DMF (10 mL) was added diethyl malonate (0.58 mL, 3.812 mmol) at room temperature under N₂ atmosphere. To this solution was added K₂CO₃ (526 mg, 3.812 mmol) followed by catalytic amounts of tetrabutylammonium hydrogen sulfate (TBAHS, 43 mg, 0.127 mmol). The reaction mixture was stirred at 75-80° C. for 48 hr and TLC analysis suggested complete conversion of starting material. After completion, the reaction mixture was quenched with ice water (10 mL) and the product was extracted using ethyl acetate (2×25 mL). The combined organic layer was washed with water (20 mL) and the organic phase was concentrated under reduced pressure to obtain compound 2 as gummy oil (crude yield 700 mg). ¹H NMR (400 MHz, DMSO-d6): δ 5.61 (s, 1H), 4.0-4.20 (m, 4H), 3.50-3.42 (m, 1H), 2.43-2.30 (m, 2H), 2.27-2.10 (m, 2H), 2.11-1.90 (m, 3H), 1.89-1.70 (m, 2H), 1.62-0.80 (m, 27H), 0.63 (s, 3H) ppm. Mass analysis: m/z 473.40 [M+H]⁺ was observed.

### Hydrolysis of Compound 2:

To a stirred solution of compound 2 (12 g, 25.38 mmol) in ethanol (120 mL) was added aq. potassium hydroxide solution (7.06 g in 120 mL water, 0.127 mol) at room temperature. The reaction mixture was heated to reflux for 2 hr and TLC analysis showed complete conversion of starting material. The ethanol was evaporated under reduced pressure and the solution was diluted with water (60 mL). The mixture was washed with DCM (60 mL, to remove impurities) and the pH of the aq. layer was adjusted to ˜2 by using 6N HCl. The product was extracted using EtOAc (2×50 mL) and concentrated to dryness to afford compound 3 as a yellow solid (9.5 g).

### Decarboxylation of Compound 3:

To a 50 mL single neck round bottom flask was added compound 3 (1 g, 2.4 mmol.) in o-xylene (5 mL). The mixture was heated to reflux for 18 h and TLC analysis showed complete conversion of starting material. o-Xylene was removed under vacuum and the residue was treated with petroleum ether and the solid was filtered. The wet cake was washed with petroleum ether and dried under vacuum to afford KCEA as an off-white solid (0.5 g). ¹H NMR (400 MHz, DMSO-D6): δ 11.95 (bs, 1H), 5.62 (s, 1H), 2.44-2.34 (m, 2H), 2.28-2.06 (m, 5H), 2.0-1.91 (m, 2H), 1.87-1.74 (m, 2H), 1.72-0.81 (m, 20H), 0.69 (s, 3H) ppm.

### Example 2. Preparation of 3,7-DKCA from KCEA

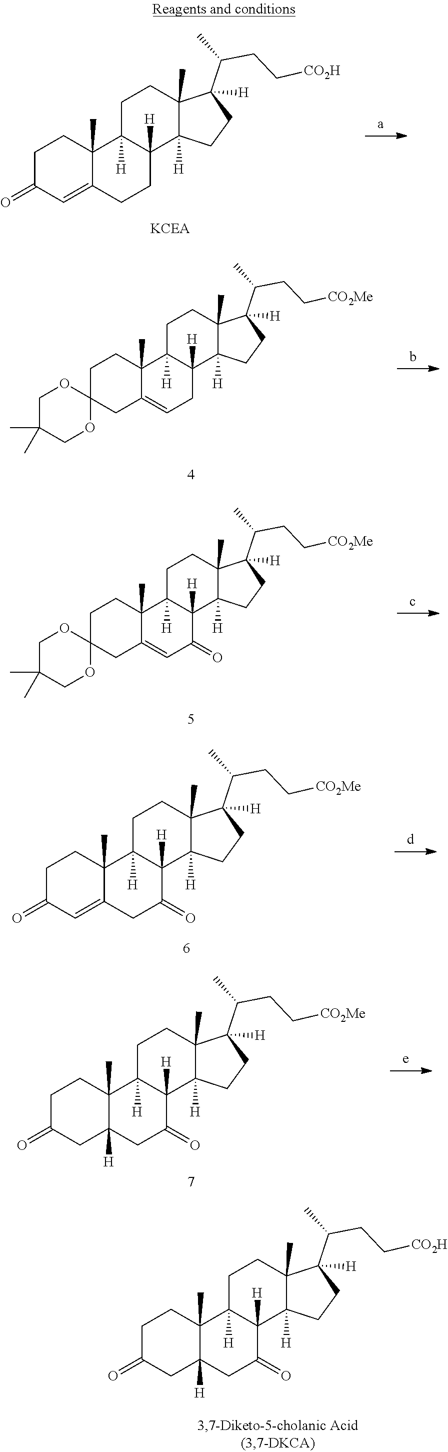

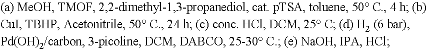

Preparation of Compound 4 from KCEA:

A 250 mL round bottom flask equipped with a stirring bar and reflux condenser was charged with toluene (90 mL), methanol (10 mL) and KCEA (10 g, 26.842 mmol). The resulting solution was inerted with nitrogen and then trimethyl orthoformate (8.8 mL, 3 equiv.) and p-toluenesulfonic acid (0.5 g, 0.1 equiv.) were added sequentially. The resulting mixture was stirred at 50-55° C. for 1 hr. The pressure was then reduced and −20 mL of solvent was removed via distillation. 2,2-Dimethylpropane-1,3-diol (22.3 g, 8 equiv.) and p-toluenesulfonic acid (0.5 g, 0.1 equiv.) were added and the reaction was continued for another 3 hr. At this point the mixture was cooled to 5° C. in an ice bath and treated with aqueous sodium acetate solution (30 g in 150 mL water). The mixture was stirred for 1 h at 5° C. and the resulting suspension was filtered to obtain crude product. This was purified further by silica gel chromatography to obtain Compound 4 as a white solid. (7.4 g). ¹H NMR (400 MHz, CDCl3) δ 5.38-5.33 (m, 1H), 3.68 (s, 3H), 3.60, 3.50 (ABq, 2H, J_{AB}=11.2 Hz), 3.49-3.43 (m, 2h), 2.61-0.91 (m, 37H), 0.69 (s, 3H); ESIMS for C₃₀H₄₈O₄ m/z 473.6 [M+H]⁺.

### Oxidation of Compound 4 to Compound 5:

To a solution of compound 4 (10 g) in 4:1 acetone/DCM (200 mL) at 25-35° C. was added N-hydroxyphthalimide (NHPI, 1.73 g), benzoyl peroxide (0.05 g), copper iodide (CuI, 0.04 g) and water (0.4 mL). The mixture was heated to 40-45° C. and air was bubbled through the mixture for 7 hr. The mixture was then cooled to 25-30° C. and the air bubbling was replaced with 98% oxygen bubbling. GC analysis after 36 hr total time indicated only 1.5% of compound 4 remains.

The reaction mixture was concentrated to a residue under vacuum and diluted with DCM (20 mL). The resulting slurry was filtered to remove NHPI. The filtrate was concentrated to −15 mL and solvent was swapped with MeOH using vacuum distillation. The mixture was diluted with MeOH (25 mL), cooled to 5-10° C. and filtered. The filter cake was washed with cold MeOH (5 mL) and dried under vacuum at 40-45° C. to afford 7.9 g of compound 5 as a light-green solid.

### Hydrolysis of Compound 5 to Compound 6:

To a solution of compound 5 (5 g) in DCM (75 mL) at 10-15° C. was added 32% conc. HCl (25 mL). The mixture was allowed to warm to 25-30° C. and held for 1.5 hr. Then the reaction mixture was diluted with water (50 mL) and the phases were separated. The aqueous layer was extracted with DCM (25 mL) and the combined DCM phases washed with water (25 mL). The DCM was treated with activated carbon (0.25 g), held for 0.25 hr and filtered over filter-aid. The filter-aid cake was washed with DCM (15 mL) and concentrated to 5-10 mL under vacuum. The residue was diluted with n-heptane (25 mL) and concentrated again to 5-10 mL. The resulting mixture was diluted with n-heptane (25 mL), cooled to 5-10° C., held for 0.5 hr and filtered. The filter cake was washed with cold n-heptane (2.5 mL) and dried under vacuum at 40-45° C. to afford 3.8 g of compound 6 as a light-orange solid.

### Hydrogenation of Compound 6 to Compound 7:

Compound 6 (180 g), dichloromethane (DCM; 45 mL) and 3-picoline (1035 mL) were combined in a 2-liter autoclave. Diazabicyclo[2.2.2]octane (DABCO; 50.4 g) and 20% Pd(OH)₂ (50% water-wet, 7.2 g) were added. The resulting mixture was stirred at 26° C. under hydrogen gas at 6 bar pressure for 22 hr. The catalyst was then removed by filtration. The solid catalyst was washed with DCM (720 mL) and the filtrate was concentrated under vacuum to remove DCM. Water (1000 mL) was added and the mixture was concentrated under vacuum at 60° C. until the total volume was ˜360 mL. Toluene (1300 mL) was added the resulting solution was washed twice with 3N HCl (2×630 mL). The aqueous washes are combined and extracted with toluene (500 mL).

The toluene fractions were combined and washed with 3N HCl (255 mL) and then distilled under vacuum to ˜360 mL. 10% Aqueous ethanol (900 mL) was added and the solution was concentrated under vacuum to ˜360 mL. Additional 10% aqueous ethanol (900 mL) was added and again the mixture was concentrated under vacuum to ˜360 mL. Additional 10% aqueous ethanol (680 mL) was added and the mixture was cooled to 0-5° C. The slurry was filtered and the cake was washed with chilled (5-10° C.) 10% aqueous ethanol (85 mL). The cake was then dried under vacuum at 40-45° C. to provide 132.4 g (73.2% yield) of compound 7 as an off-white solid.

The solid was combined with additional lots of compound 7 to give 257 g. This was dissolved in DCM (514 mL) and 10% aqueous ethanol (1030 mL) was added. The resulting mixture was distilled under vacuum to a volume of ˜500 mL, and then additional 10% aqueous ethanol (1030 mL) was added. After concentrating under vacuum again to ˜500 mL, additional 10% aqueous ethanol (1030 mL) was added. The mixture was cooled to 0-5° C. and filtered, and the cake was washed with chilled (5-10° C.) 10% aqueous ethanol (125 mL). The cake was then dried under vacuum at 40-45° C. to provide 238 g (92.6% recovery) of compound 7 as an off-white solid.

Melting point=167° C.; Purity by CAD HPLC (w/w %)=98.7% (5α-impurity=0.37%); ¹H-NMR (400 MHz, CDCl₃): δ 3.67 (s, 3H), 2.90 (dd, J=12.8 & 6.5 Hz, 1H), 2.49 (t, J=11.4 Hz, 1H), 0.95-2.40 (m, 27H), 0.93 (d, J=6.4 Hz, 3H), 0.69 (s, 3H).

### Hydrolysis of Compound 7 to 3,7-DKCA:

To a solution of compound 7 (6 g) in IPA (30 mL/g) was added a solution of NaOH (1.5 g, 2.5 equiv) in water (30 mL) at room temperature. The reaction was warmed to 55-60° C. until it was found to be complete by TLC analysis.

The reaction mixture was concentrated to ˜30 mL to remove residual IPA and the resulting aqueous solution washed with MTBE (2×30 mL). The aqueous phase was acidified to pH 2 using 6 M HCl, leading to the formation of a slurry. After cooling to 10-15° C., the slurry was filtered, washed with water and dried under vacuum at 45-50° C. to afford 4.2 g of 3,7-DKCA as a light-brown solid.

This material can be used as the starting material for Example 13.

### Example 3. Preparation of TUDCA from 3,7-DKCA (7-Ketone Reduction First)

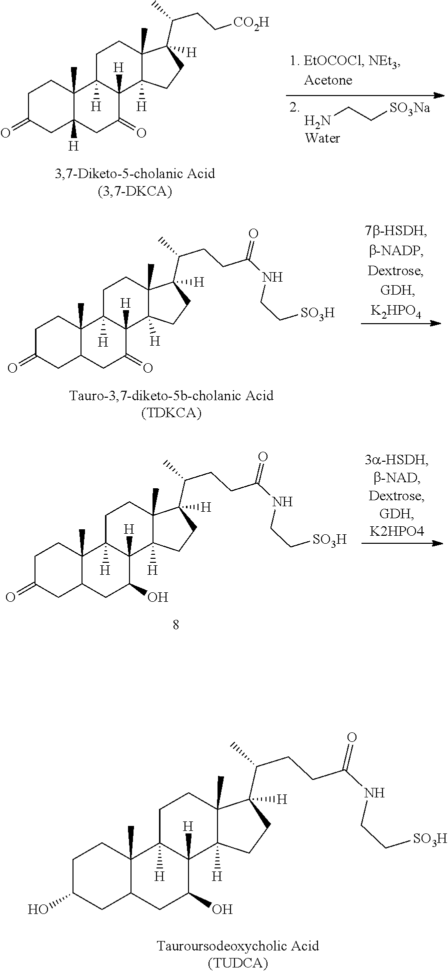

### Conversion of 3,7-DKCA to TDKCA:

To a solution of 3,7-DKCA (5 g) and triethylamine (1.75 mL, 0.97 equiv) in acetone (30 mL) at 0-5° C. was added ethyl chloroformate (1.2 mL, 0.97 equiv). The mixture was warmed to room temperature and held at this temperature until it was determined by TLC to be complete. The reaction mixture was filtered and the resulting filtrate was added dropwise to a mixture of taurine (1.93 g, 1.2 eq) and NaOH (0.62 g, 1.2 eq) in water (3.5 mL) at room temperature. The reaction mixture was held at this temperature until it was determined by TLC to be complete.

Conc. HCl (˜1.5 mL) was added to the reaction mixture until the pH was ˜1. The mixture was held for 1 h at room temperature and filtered. The filtrate was diluted with acetone (75 mL) and the resulting slurry was held for ½ hr at room temperature and filtered. The solids were washed with acetone (10 mL) and dried under vacuum to afford 4.5 g of TDKCA as a white solid. ¹H NMR (400 MHz, DMSO-D6): δ 7.64 (t, J=5.2 Hz, 1H), 3.27 (dd, J=13.6 & 6 Hz, 2H), 2.92 (dd, J=12.8 & 5.2 Hz), 2.58-2.48 (m, 2H), 2.43-2.30 (m, 1H), 2.25-0.90 (m, 27H), 0.87 (d, J=4.8 Hz, 3H), 0.64 (s, 3H);

Mass: m/z 494.77 [M−H⁺]

### Selective Reduction of the 7-Ketone of TDKCA to Compound 8:

To a 100 mL single neck round bottom flask was added TDKCA (1 g, 2.02 mmol), dextrose (1 g) and 3-NADP (33 mg) in 250 mM K₂HPO₄ buffer (65 mL) at room temperature. The mixture was stirred for 0.5 h to get a clear solution. To this solution 73-HSDH (66 mg) and GDH (3.3 mg) were added and the reaction mixture was stirred for 18 h at room temperature and TLC analysis showed complete conversion of starting material.

The mixture was acidified using 6N HCl to pH-1 and stirred for 1 hr. The product was extracted with n-BuOH (2×25 mL). The organic layers were combined and concentrated under vacuum until ˜3 mL of solvent remained. The slurry was diluted with acetone (30 mL) and stirred for 14 hr. The resulting slurry was filtered, washed with acetone and dried under vacuum to obtain 0.58 g of compound 8 as an off-white solid.

¹H NMR (400 MHz, DMSO-D6): δ 7.70 (bs, 1H), 3.40-3.30 (m, 2H), 3.30-3.21 (m, 2H), 2.63 (t, J=14 Hz, 1H), 2.55 (t, J=6 Hz, 2H), 2.38-2.26 (m, 1H), 2.12-0.98 (m, 27H), 0.96 (s, 3H), 0.88 (d, J=6.4 Hz, 3H), 0.64 (s, 3H);

Mass: m/z 498.43 [M+H⁺]

### Selective Reduction of the 3-Ketone of Compound 8 to Provide TUDCA:

To a 250 mL single neck round bottom flask were added compound 8 (1 g, 2.01 mmol), dextrose (1.3 g), β-NAD (33 mg) and 250 mM K₂HPO₄ buffer (70 mL) at room temperature. The mixture was stirred for 0.5 h to get a clear solution. 3α-HSDH (66 mg) and GDH (2 mg) were added and the resulting mixture stirred for 20 hr at room temperature. TLC analysis is expected to show complete conversion of starting material.

The reaction mixture was quenched with 2N HCl solution until the pH reached ˜1, and then the product extracted with butanol (3×25 mL). The organic fractions were combined and concentrated to ˜3 mL. The resulting mixture was diluted with acetone (30 mL) and stirring continued for 15 hr. The resulting slurry was filtered to obtain TUDCA as a white solid.

¹H NMR (400 MHz, CD₃OD): δ 3.67 (t, J=6.8 Hz, 2H), 3.56-3.46 (m, 1H), 3.02 (t, J=6.8 Hz, 2H), 2.37-2.33 (m, 1H), 2.23-2.33 (m, 1H), 1.13-0.95 (m, 33H), 0.74 (s, 3H); Mass: m/z 498.78 [M−H⁺]

### Example 4. Preparation of TUDCA from 3,7-DKCA (3-Ketone Reduction First)

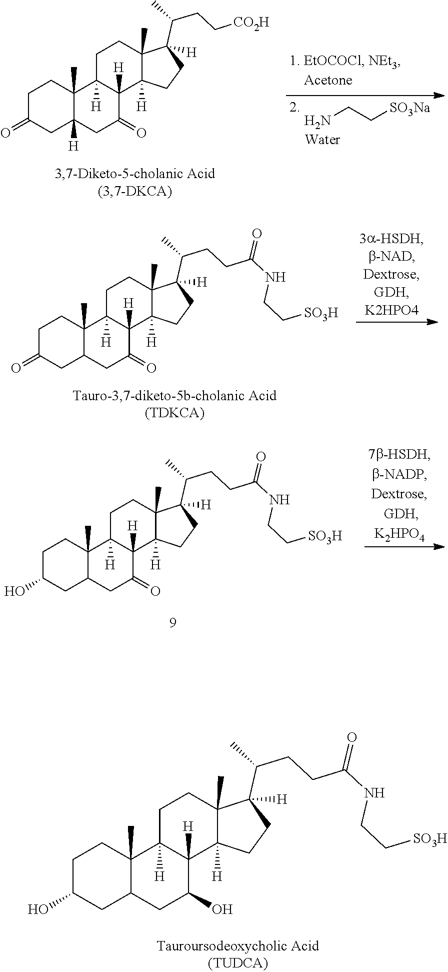

### Selective Reduction of the 3-Ketone of TDKCA to Compound 9:

To a 250 mL single neck round bottom flask was added TDKCA (5 g, 10.1 mmol), along with 250 mM K₂HPO₄ buffer (200 mL) at room temperature. The pH was adjusted to 8.2 by adding 1M KOH (0.95 mL) and the mixture was stirred for 0.25 h to get a clear solution. To this solution were added dextrose (6.5 g) and β-NAD (200 mg) and the reaction mixture stirred for 15 min. The pH was again adjusted to 8.2 using 1M KOH solution (0.03 mL). 7β-HSDH (165 mg) and GDH (50 mg) were added. The pH was maintained at 8 by periodic addition of 1 M KOH. The reaction mixture was stirred at room temperature until TLC analysis showed complete consumption of starting material.

The mixture was then acidified with 6N HCl to pH-1 and stirred for 1 hr. The product was extracted with n-BuOH (3×25 mL). The organic layers were combined and washed with water. The solvent was removed under vacuum to provide a gummy solid. The solid was dissolved in in a mixture of water (1.5 mL) and acetone (1.5 mL) at 60° C. The solution was cooled to RT and stored at 4° C. for 12 h and the resulting solid was filtered. The wet cake was washed with cold water and dried under vacuum to obtain 1.7 g of compound 9 as an off-white solid.

¹H NMR (400 MHz, DMSO-D6): δ 7.70 (bs, 1H), 3.35-3.30 (m, 1H), 3.30-3.25 (m, 2H), 2.90 (dd, J=12.4 & 6 Hz), 2.54 (t, J=7.2 Hz), 2.44 (t, J=11.2 Hz), 2.12-0.82 (m, 28H), 0.87 (d, J=6.4 Hz), 0.61 (s, 3H);

Mass: m/z 496.72 [M−H⁺]

### Selective Reduction of the 7-Ketone of Compound 9 to Provide TUDCA:

To a 250 mL single neck round bottom flask were added compound 9 (1.5 g, 3.02 mmol), and 250 mM K₂HPO₄ buffer (105 mL) at room temperature. The mixture was stirred for 0.5 h to get a clear solution. Dextrose (1.95 g), β-NADP (85.7 mg) were added and the pH was adjusted to 8.2 by the addition of 1M KOH. 7β-HSDH (120 mg) and GDH (6.42 mg) were added and the resulting mixture was stirred for 20 hr at room temperature. The pH was maintained at 8 by the periodic addition of 1M KOH. TLC analysis showed complete conversion of starting material.

The mixture was then acidified with 6N HCl (1.6 mL) to pH-1 and stirred for 1 hr. The product was extracted with n-BuOH (2×10 mL). The organic fractions were combined and washed with water (10 mL) and then concentrated to dryness to obtain a gummy residue (950 mg). This solid was dissolved by heating to 60° C. in 1-2 mL of water. The solution was then cooled to 4° C. and held for 24 hr. The resulting slurry was filtered and dried to obtain TUDCA as a white solid. Yield=850 mg.

¹H NMR (400 MHz, CD₃OD): δ 3.66 (t, J=6.8 Hz, 2H), 3.54 (m, 1H), 3.02 (t, J=6.8 Hz, 2H), 2.37-2.33 (m, 1H), 2.23-2.33 (m, 1H), 1.13-0.95 (m, 33H), 0.75 (s, 3H);

Mass: m/z 498.67 [M−H⁺]

### General Methods for Examples 5-9

Isolation, handling and manipulation of DNA are carried out using standard methods (Green and Sambrook, 2012), which includes digestion with restriction enzymes, PCR, cloning techniques and transformation of bacterial cells.

Synthetic DNA is ordered from a commercial vendor, such as Eurofins, IDT, Genewiz or Twist Biosciences, as described in the examples. Genes are to be supplied in custom vectors or as linear DNA fragments, as described in the examples.

### Media

2TY medium contains 16 g/L bacto-tryptone, 10 g/L yeast extract and 5 g/L NaCl and is sterilised by autoclaving. 2TY agar additionally contains 15 g/L agar.

Low-salt LB contains 10 g/L tryptone, 5 g/L yeast extract and 5 g/L NaCl.

Seed medium contains 3 g/L yeast extract, 2.5 g/L dibasic potassium phosphate, 18 g/L vegetable peptone, 5 g/L NaCl and 10 g/L glucose.

Fermentation medium contains yeast extract 5 g/L, ammonium sulfate 1.7 g/L, dibasic potassium phosphate 7 g/L, citric acid 1 g/L, iron chloride 0.04 g/L, calcium chloride 0.03 g/L, magnesium sulfate 4.6 g/L, copper chloride 0.05 mg/L, boric acid 0.025 mg/L sodium iodide 0.5 mg/L manganese sulfate 0.5 mg/L zinc sulfate 0.1 mg/L and sodium molybdate 0.1 mg/L

Fermentation substrate feed medium contains yeast extract 5 g/L, ammonium sulfate 1.7 g/L, dibasic potassium phosphate 7 g/L, citric acid 1 g/L, iron chloride 0.04 g/L, calcium chloride 0.03 g/L, magnesium sulfate 4.6 g/L, copper chloride 0.05 mg/L, boric acid 0.025 mg/L sodium iodide 0.5 mg/L manganese sulfate 0.5 mg/L zinc sulfate 0.1 mg/L sodium molybdate 0.1 mg/L and 350 g/L glucose

### Materials

Restriction enzymes are purchased from New England Biolabs (NEB) or Promega. Media components, chemicals and PCR primers are obtained from Sigma-Aldrich (Merck).

### Example 5. Construction of an Escherichia coli Strain Capable of Expressing a Gene Encoding a 3α-Hydroxy-Steroid Dehydrogenase Enzyme from Comamonas Testosterone

Plasmid pSAND150 was constructed as follows. SEQ ID NO. 1 was ordered as synthetic DNA (Integrated DNA Technologies) and amplified by PCR using primers SEQ ID NO. 2 and SEQ ID NO. 3, resulting in a 2541 bp fragment, to be used as fragment A. SEQ ID NO. 4 was ordered as synthetic DNA (Integrated DNA technologies) and amplified by PCR using primers SEQ ID NO. 5 and SEQ ID NO. 6, resulting in a 2927 bp fragment, to be used as fragment B. Fragment A was inserted into PCR-amplified fragment B using the SLiCE cloning method (Zhang et al., 2014), forming plasmid pSAND150. Correct assembly of the plasmid was verified by restriction digest and by sanger sequencing using primers SEQ ID NO. 7, SEQ ID NO. 8 and SEQ ID NO. 9.

Plasmid pSAND151, to express a gene encoding a 3α-hydroxy-steroid dehydrogenase from Comamonas testosteroni, was constructed as follows. Plasmid pSAND150 was amplified by PCR using primers SEQ ID NO. 10 and SEQ ID NO. 11, followed by digestion with restriction enzyme DpnI, to be used as the plasmid backbone. SEQ ID NO. 12 was ordered as synthetic DNA (Integrated DNA technologies) and amplified by PCR using primers SEQ ID NO. 13 and SEQ ID NO. 14. The resulting 874 bp fragment was inserted into PCR-amplified pSAND150 using the SLiCE cloning method (Zhang et al., 2014), forming plasmid pSAND151. Correct assembly of plasmid pSAND151 was verified by colony PCR primers SEQ ID NO. 7 and SEQ ID NO. 15 and by sanger sequencing using primers SEQ ID NO. 7, SEQ ID NO. 9 and SEQ ID NO. 15.

Plasmid pSAND151 was used to transform E. coli BL21(DE3) by electroporation using standard methods. The resulting strain was labelled Escherichia coli sp. SAND150.

### Example 6. Production of a 3α-Hydroxy-Steroid Dehydrogenase Enzyme

50 mL low-salt LB medium containing 12.5 g/mL kanamycin in a 250-mL baffled Erlenmeyer flask was inoculated with E. coli sp. SAND150 and incubated at 37° C. with shaking at 250 RPM, 2.5 cm throw for 18 hours, to be used as the preculture.

4 mL of preculture was transferred to a 2 Litre baffled glass Erlenmeyer flask with 400 mL Seed medium containing kanamycin at 12.5 pg/mL. This culture was incubated at a 37° C. and rotated at 250 rpm for 7 hours, to be used as the seed culture. The cell density reached 7.03 OD₆₀₀.

60 mL seed culture was transferred to a 6.4 litre production stage bioreactor containing 2 litres of Fermentation media described in media section to achieve a starting biomass of 0.2 OD₆₀₀. The bioreactor was operated as a fed-batch variable volume fermentation at 31% to 78% volumetric space efficiency. The fermentation temperature was controlled to a constant 30° C. until induction with no back pressure. Dissolved oxygen was controlled at 30% with a control statement increasing stirrer incrementally from 200 to 1200 rpm increasing by 25 rpm when PO2 drops below setpoint activated at 10-minute intervals and a fixed manual airflow of 4 litres of air per minute. The agitation was achieved by two conventional 6-flat bladed disc turbines and the airflow was sparged via a submerged sparger. pH was controlled at 7.2 with the automatic addition of 28% ammonium hydroxide. Fermentation substrate feed was applied to the fermenter from the start of inoculation, where it received a linear rate of 19.2 mL/hr to 103.1 mL/hr over 24 hours.

The linear feed was continued until the optical density reached 59.8 OD₆₀₀ and the culture was induced by the addition of 0.5 mM Isopropyl B-D-1-thiogalactopyranoside (IPTG) and reduction of temperature to 25° C. The substrate feed rate was then switched to an event-based feeding method for the remainder of the production, adding 9 mL shot of feed when the dissolved oxygen rose above 30%. The fermentation was harvested once 22.5 hours had passed since induction. Fermentation broth was centrifuged at 8000 rcf at 4° C., 45 minutes and 884 g of cell pellet was frozen at −80° C. Cells solids were then resuspended in 50 mM potassium phosphate buffer pH 8.0 to a concentration of 40% solids. The slurry was then mechanically lysed using a french press cell disruptor at 1500 psi with 3 passes. Bulk lysate was diluted to 3.2 litres before polyethyleneimine was added to a final concentration of 0.4%. The mixture was agitated for 10 minutes before centrifuged again at 8000×g for 15 minutes. The supernatant was retained, and the volume was concentrated by 37% using a 5 kDa MWCO PES filtration membrane. Retentate liquid was then dried under vacuum to create a lyophilised powder.

### Example 7. Construction of an Escherichia coli Strain Lacking Native 7α-Hydroxysteroid Dehydrogenase Activity

Plasmid pSAND152, to interrupt the hdhA gene in E. coli, was constructed as follows. SEQ ID NO. 16 was ordered as circular synthetic DNA (Twist Bioscience) and cleaved with restriction enzymes BsrGI and XbaI, to be used as the plasmid backbone. SEQ ID NO. 17 was ordered as synthetic DNA (Integrated DNA technologies) and amplified by PCR using primers SEQ ID NO. 20 and SEQ ID NO. 21. The resulting 364 bp fragment was digested with restriction enzymes BsrGI and XbaI. The digested synthetic DNA was inserted into the cleaved plasmid backbone by ligation following standard methods, forming plasmid pSAND152. Transformants were plated onto 2TY agar containing 34 g/mL chloramphenicol. Correct assembly of plasmid pSAND152 was confirmed by sanger sequencing using primers SEQ ID NO. 18 and SEQ ID NO. 19.

Plasmid pSAND152 was used to transform E. coli BL21(DE3) by electroporation using standard methods and plated onto 2TY agar containing 50 g/mL kanamycin and 1 mM IPTG. Agar plates were incubated at 30° C. for approximately 18 hours, followed by incubation at ambient temperature for a further 3 days. Disruption of the hdhA gene was verified by growth on 2TY agar plates containing either 50 g/mL kanamycin or 34 g/mL chloramphenicol, where kanamycin resistance and chloramphenicol sensitivity indicates successful disruption.

Disruption of the hdhA gene was further verified as follows. A 2829 bp DNA fragment was amplified by PCR from the genome of the transformant using primers SEQ ID NO. 22 and SEQ ID NO. 23. The amplified DNA fragment was subsequently sequenced using primers SEQ ID NO. 22 and SEQ ID NO. 23. The resulting strain was labelled Escherichia cob sp. SAND151.

### Example 8. Construction of an Escherichia coli Strain Capable of Expressing a Gene Encoding an Engineered 7β-Hydroxy-Steroid Dehydrogenase Enzyme from Ruminococcus Torques

Plasmid pSAND153, to express a gene encoding a 7β-hydroxy-steroid dehydrogenase, was constructed as follows. Plasmid pSAND150 was amplified by PCR using primers SEQ ID NO. 10 and SEQ ID NO. 11, followed by digestion with restriction enzyme DpnI, to be used as the plasmid backbone.

SEQ ID NO. 24 was ordered as synthetic DNA (Integrated DNA technologies) and amplified by PCR using primers SEQ ID NO. 25 and SEQ ID NO. 26. The resulting 895 bp fragment was inserted into PCR-amplified pSAND150 using the SLiCE cloning method (Zhang et al., 2014), forming plasmid pSAND153. Correct assembly of plasmid pSAND153 was verified by colony PCR and by Sanger sequencing using primers SEQ ID NO. 7, SEQ ID NO. 9 and SEQ ID NO. 15.

Plasmid pSAND154, to express a gene encoding a 7β-hydroxy-steroid dehydrogenase, was constructed as follows. Plasmid pSAND153 was amplified by PCR using primers SEQ ID NO. 27 and SEQ ID NO. 28, to be used as the plasmid backbone.

SEQ ID NO. 29 was ordered as synthetic DNA (Integrated DNA Technologies) and amplified by PCR using primers SEQ ID NO. 30 and SEQ ID NO. 31. The resulting 1066 bp fragment was inserted into PCR-amplified pSAND154 using the SLiCE cloning method (Zhang et al., 2014), forming plasmid pSAND154.

Plasmid pSAND154 was used to transform E. coli sp. SAND151 by electroporation using standard methods. The resulting strain was labelled Escherichia coli sp. SAND152.

### Example 9. Production of a 7β-Hydroxy-Steroid Dehydrogenase Enzyme

50 mL low-salt LB medium containing 12.5 g/mL kanamycin in a 250-mL baffled Erlenmeyer flask was inoculated with E. coli sp. SAND150 and incubated at 37° C. with shaking at 250 RPM, 2.5 cm throw for 18 hours, to be used as the preculture.

4 mL of preculture was transferred to a 2 Litre baffled glass Erlenmeyer flask with 400 mL Seed medium containing kanamycin at 12.5 pg/mL. This culture was incubated at a 37° C. and rotated at 250 rpm for 7 hours, to be used as the seed culture. The cell density reached 4.8 OD₆₀₀.

60 mL seed culture was transferred to a 6.4 litre production stage bioreactor containing 2 litres of Fermentation media described in media section to achieve a starting biomass of 0.14 OD₆₀₀. The bioreactor was operated as a fed-batch variable volume fermentation at 31% to 78% volumetric space efficiency. The fermentation temperature was controlled to a constant 30° C. until induction with no back pressure. Dissolved oxygen was controlled at 30% with a control statement increasing stirrer incrementally from 200 to 1200 rpm increasing by 25 rpm when PO2 drops below setpoint activated at 10-minute intervals and a fixed manual airflow of 4 litres of air per minute. The agitation was achieved by two conventional 6-flat bladed disc turbines and the airflow was sparged via a submerged sparger. pH was controlled at 7.2 with the automatic addition of 28% ammonium hydroxide. Fermentation substrate feed was applied to the fermenter from the start of inoculation, where it received a linear rate of 19.2 mL/hr to 103.1 mL/hr over 24 hours.

The linear feed was continued until the optical density reached 70 OD₆₀₀ and the culture was induced by the addition of 0.5 mM Isopropyl B-D-1-thiogalactopyranoside (IPTG) and reduction of temperature to 25° C. The substrate feed rate was then switched to an event-based feeding method for the remainder of the production, adding 9 mL shot of feed when the dissolved oxygen rose above 30%. The fermentation was harvested once 20 hours had passed since induction. Fermentation broth was centrifuged at 8000 rcf at 4° C., 45 minutes and 751 g of cell pellet was frozen at −80° C. Cells solids were then resuspended in 50 mM potassium phosphate buffer pH 8.0 to a concentration of 30% solids. The slurry was then mechanically lysed using a french press cell disruptor at 1500 psi with 3 passes. Polyethyleneimine was added to the bulk homogenised lysate to a final concentration of 0.8% and agitated for 10 minutes before being centrifuged again at 8000×g for 30 minutes. The supernatant was retained, and the volume was concentrated by 50% using a 10 kDa MWCO PES filtration membrane. Retentate liquid was then dried under vacuum to create a lyophilised powder.

### Example 10. TUDCA Salts and Crystalline Characterization

### 10.1 Pattern 1-A (L-Arginine)

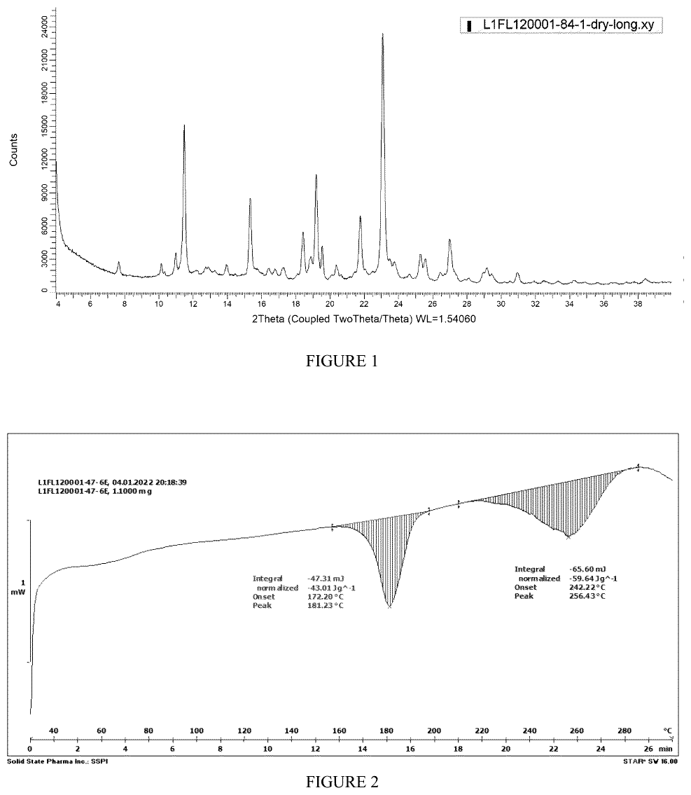

Pattern 1-A (L-arginine) was prepared by slurrying commercial grade plant derived TUDCA in 13 mL of IPA:MeOH (7:3 vol.) at 55° C. for 2 h followed by stirring at RT overnight. The sample was sonicated for 30 min before stirring at 55° C. for 2 h. Seeding was done after sonication by solid L-arginine TUDCA. The slurry was then filtered and dried at 50° C. at −29 in Hg overnight. A yield of 416.49 mg (86.3% w/w) was isolated. Characterization data for Pattern 1-A (L-arginine TUDCA) are summarized in Table 1 and Table 2 and depicted in FIG. 1 and FIG. 2.

TABLE 1
Characterization of Pattern 1-A (L-arginine).

| Description | Results |
|---|---|
| DSC onsets (° C.) | 146.64 (shoulder), |
| | 168.06, 234.83 |
| TGA mass loss (wt. %) | 1.18, 1.13, 5.52 (likely |
| | decomposition) |
| DVS mass change 2-80% | 2.91 |
| RH (wt. %) | |
| NMR residual solvent | 1.65 (IPA) |
| (wt. %) | |
| Counter ion stoichiometry | 1.00:0.97 |
| (API:CI) | |
| HPLC chemical purity | 99.89 |
| (area %) | |
| Morphology | Small needles |

TABLE 2
XRPD peak table for solid Pattern 1-A (L-arginine).

| Angle | | Relative |
|---|---|---|
| (°2θ) | d-Spacing (Å) | intensity |
| 7.66 | 11.53 | 5 |
| 10.14 | 8.71 | 5 |
| 10.34 | 8.55 | 2 |
| 10.99 | 8.04 | 9 |
| 11.48 | 7.70 | 61 |
| 12.19 | 7.25 | 2 |
| 12.73 | 6.95 | 3 |
| 12.88 | 6.87 | 3 |
| 13.26 | 6.67 | 2 |
| 13.96 | 6.34 | 5 |
| 15.34 | 5.77 | 32 |
| 15.74 | 5.62 | 3 |
| 16.00 | 5.53 | 2 |
| 16.42 | 5.39 | 4 |
| 16.78 | 5.28 | 4 |
| 17.25 | 5.14 | 4 |
| 18.09 | 4.90 | 2 |
| 18.43 | 4.81 | 19 |
| 18.89 | 4.69 | 9 |
| 19.19 | 4.62 | 43 |
| 19.54 | 4.54 | 13 |
| 20.09 | 4.42 | 2 |
| 20.35 | 4.36 | 6 |
| 20.65 | 4.30 | 2 |
| 21.44 | 4.14 | 3 |
| 21.77 | 4.08 | 26 |
| 22.08 | 4.02 | 4 |
| 22.51 | 3.95 | 3 |
| 23.08 | 3.85 | 100 |
| 23.50 | 3.78 | 8 |
| 23.77 | 3.74 | 7 |
| 24.66 | 3.61 | 2 |
| 25.29 | 3.52 | 10 |
| 25.57 | 3.48 | 9 |
| 26.45 | 3.37 | 3 |
| 26.99 | 3.30 | 17 |
| 27.34 | 3.26 | 3 |
| 28.12 | 3.17 | 2 |
| 28.94 | 3.08 | 4 |
| 29.18 | 3.06 | 6 |
| 29.45 | 3.03 | 4 |
| 30.96 | 2.89 | 4 |
| 38.42 | 2.34 | 2 |

Note.
The peak cut off used was <2% relative intensity.

### 10.2 Pattern 5-A (L-lysine)

Pattern 5-A (L-lysine) was produced by slurrying L-lysine and commercial grade plant derived TUDCA in 2.5 mL of ACN:MeOH (1:1 vol.) at 60° C. for 2 h at 400 rpm followed by stirring at RT overnight. Sonication was done for 2 h before stirring at 60° C. The slurry was seeded before and after sonication with solid L-lysine TUDCA. The sample was filtered and dried at 50° C. and −29 in Hg for 3 h. Temperature cycling was carried out in an attempt to obtain more crystalline material. FIG. 3 shows the XRPD diffractogram of Pattern 5-A (L-lysine TUDCA). XRPD data are reported in Table 3.

TABLE 3
XRPD peak table for solid Pattern 5-A (L-lysine).

| Angle | d-Spacing | Relative intensity |
|---|---|---|
| (°2θ) | (Å) | % |
| 6.92 | 12.76 | 8 |
| 8.74 | 10.11 | 100 |
| 10.38 | 8.51 | 10 |
| 12.24 | 7.23 | 9 |
| 17.25 | 5.14 | 9 |
| 20.05 | 4.43 | 20 |

Note.
The peak cut off used was 5% relative intensity.

### 10.3 Pattern 6-A (L-histidine)

Pattern 6-A (L-histidine) was prepared by slurrying L-histidine and commercial grade plant derived TUDCA in 2.5 mL of THF:IPA (4:6 vol.) at 60° C. for 2 h at 400 rpm followed by stirring at RT overnight. Sonication was done for 2 h before stirring at 60° C. The slurry was seeded before and after sonication with solid L-histidine TUDCA. The sample was filtered and dried at 50° C. and −29 in Hg for 3 h. A yield of 27.6 mg (34.5% w/w) was obtained. Characterization data for Pattern 6-A (L-histidine TUDCA) are summarized in Table 4 and Table 5 and given in FIG. 4 and FIG. 5.

TABLE 4
Characterization of solid Pattern 6-A (L-histidine).

| Description | Results |
|---|---|
| XRPD result | 6-A (L-histidine) |
| DSC onsets (° C.) | 38.08, 129.34, |
| | 159.22, 199.95 |
| TGA mass loss (wt. %) | 5.75 (likely 0.42 eq. of |
| | THF) |
| HPLC chemical purity (% | 99.84 |
| a/a) | |
| Morphology | Fine particles and |
| | agglomerates |

TABLE 5
XRPD peak table for solid Pattern 6-A (L-histidine).

| Angle | d-Spacing | Relative |
|---|---|---|
| (°2θ) | (Å) | intensity % |
| 6.76 | 13.07 | 100 |
| 7.04 | 12.55 | 2 |
| 9.40 | 9.40 | 9 |
| 12.38 | 7.14 | 13 |
| 14.09 | 6.28 | 1 |
| 14.95 | 5.92 | 2 |
| 16.90 | 5.24 | 1 |
| 17.93 | 4.94 | 4 |
| 18.50 | 4.79 | 2 |
| 18.85 | 4.70 | 1 |
| 19.49 | 4.55 | 3 |
| 19.89 | 4.46 | 1 |
| 21.09 | 4.21 | 1 |
| 21.38 | 4.15 | 2 |
| 22.66 | 3.92 | 2 |
| 23.77 | 3.74 | 2 |
| 24.15 | 3.68 | 1 |
| 26.03 | 3.42 | 1 |
| 28.59 | 3.12 | 1 |

Note.
The peak cut off used was <1% relative intensity.

### Example 11. Crystalline Forms of Plant Derived TUDCA

### Pattern a (Dihydrate)

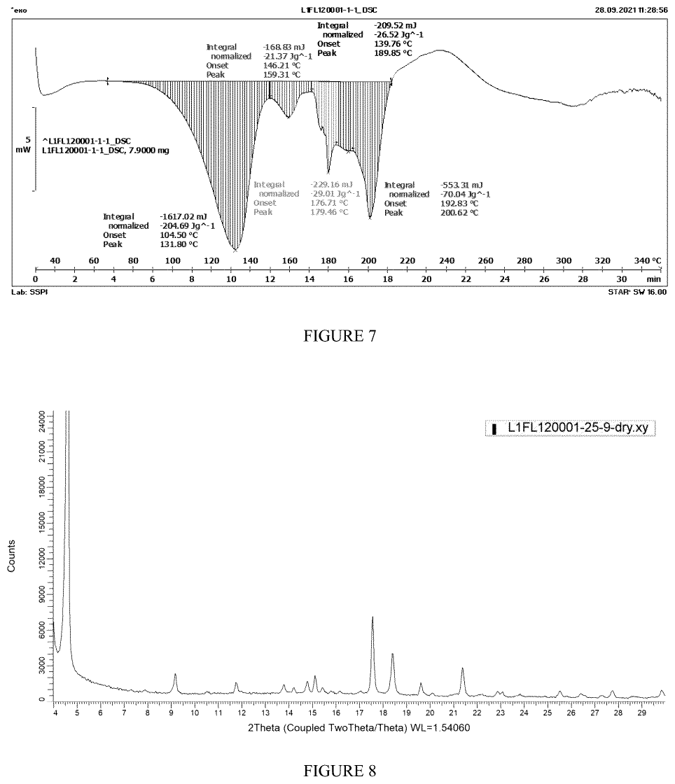

Pattern A can be prepared by the slurry method. In this experiment, 206.2 mg of TUDCA was mixed with 3 mL of acetone:water (9:1 vol.) in a 4 mL vial at RT. The mixture was stirred for 2 h at 400 rpm and filtered using filtration paper. The filtered sample was washed with 1.5 vol. of acetone:water (9:1 vol.) and analyzed by XRPD. The sample was then dried in a 50° C. oven under −29.5 in Hg overnight. The yield was 119.3 mg (58% w/w). Characterization data for commercial grade Pattern A TUDCA derived from plant sources are summarized in Table 6 and Table 7 and given in FIG. 6 and FIG. 7.

TABLE 6
Summary of characterization data from the scale-up of Pattern A.

| Description | Results | |
|---|---|---|
| XRPD result | Pattern A | |
| DSC onsets (° C.) | 90.23, 149.45, 168.77 (exo), | |
| | 173.34, 197.88, 218.33 (exo) | |
| TGA mass loss (wt. %) | 6.49 (up to 160° C.), 6.49 | |
| | (160-220° C.) | |
| Karl Fischer water content | 6.40 | (1.9 eq. water) |
| (wt. %) | | |
| NMR residual solvent | 0.5 | (0.04 eq. of acetone) |

| | |
|---|---|
| (wt. %) | |
| HPLC chemical purity (% | 99.79 |
| a/a) | |
| DVS mass change 2-95% | 0.96 |
| RH (wt. %) | |
| Morphology | Irregularly-shaped part |

TABLE 7
Peak list table for Form A.

| | | Relative intensity |
|---|---|---|
| 2θ (°) | d-spacing (Å) | % |
| 5.19 | 17.01 | 100 |
| 9.25 | 9.55 | 4 |
| 10.31 | 8.57 | 38 |
| 10.49 | 8.43 | 10 |
| 13.70 | 6.46 | 9 |
| 13.98 | 6.33 | 34 |
| 14.13 | 6.26 | 59 |
| 14.98 | 5.91 | 74 |
| 15.10 | 5.86 | 32 |
| 15.45 | 5.73 | 4 |
| 17.60 | 5.03 | 73 |
| 18.17 | 4.88 | 22 |
| 18.47 | 4.80 | 73 |
| 18.81 | 4.71 | 7 |
| 19.08 | 4.65 | 26 |
| 20.83 | 4.26 | 10 |
| 21.10 | 4.21 | 79 |
| 21.39 | 4.15 | 28 |
| 22.03 | 4.03 | 12 |
| 23.26 | 3.82 | 13 |
| 23.58 | 3.77 | 10 |
| 23.92 | 3.72 | 9 |
| 24.38 | 3.65 | 9 |
| 24.89 | 3.57 | 12 |
| 25.63 | 3.47 | 9 |
| 25.82 | 3.45 | 21 |
| 27.64 | 3.22 | 5 |
| 27.98 | 3.19 | 6 |
| 28.19 | 3.16 | 10 |
| 29.36 | 3.04 | 7 |
| 29.91 | 2.98 | 6 |
| 31.09 | 2.87 | 17 |
| 31.64 | 2.83 | 4 |
| 32.24 | 2.77 | 6 |
| 32.67 | 2.74 | 9 |
| 33.47 | 2.68 | 4 |
| 36.42 | 2.47 | 6 |
| 36.71 | 2.45 | 5 |
| 37.83 | 2.38 | 6 |
| 38.60 | 2.33 | 7 |

Note
Relative intensity of more than 3% was considered.

### Pattern L

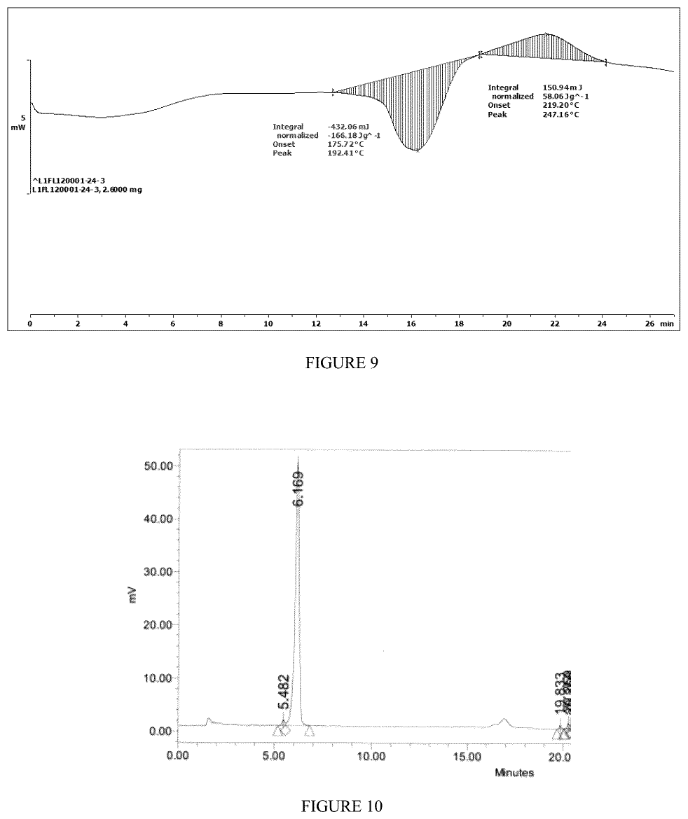

Pattern L was prepared by the slurry method. In this experiment, 205.4 mg of solid commercial-grade plant derived TUDCA was mixed with 2.2 mL of IPAc:MeOH (7.3:2.9 vol.) in a 4 mL vial at 50° C. The mixture was sonicated for 1 h and stirred for 15 min at 400 rpm, after which it was filtered and analyzed by XRPD. The sample was then dried in a 50° C. oven under −29.5 in Hg for 3 h, but the crystallinity was observed to decrease upon drying. As this pattern was a possible hydrate, it was placed in a humidity chamber at 55% RH to see if the crystallinity would increase. The yield was 114.6 mg (67.7% w/w). Characterization data for Pattern L TUDCA are summarized in Table 8 and Table 9 and given in FIG. 8 and FIG. 9.

TABLE 8
Summary of characterization data from the scale-up of Pattern L

| Description | Results |
|---|---|
| XRPD result | Pattern L |
| DSC onsets (° C.) | 173.72 |
| TGA mass loss (wt. %) | 3.1 (40-120° C.), 7.5 (120- |
| | 230° C.) |
| Karl Fischer water content | 1.71 |
| (wt. %) | |
| NMR residual solvent | 1.61 (0.08 eq. of IPAc) |
| (wt. %) | |
| Morphology | Irregularly-shaped particles |

TABLE 9
Peak list table for Pattern L.

| | d-Spacing | Relative |
|---|---|---|
| °2θ | (Å) | intensity % |
| 4.59 | 19.23 | 100 |
| 9.17 | 9.63 | 4 |
| 11.77 | 7.51 | 2 |
| 13.78 | 6.42 | 2 |
| 14.20 | 6.23 | 1 |
| 14.78 | 5.99 | 2 |
| 15.11 | 5.86 | 4 |
| 15.44 | 5.74 | 1 |
| 17.05 | 5.20 | 1 |
| 17.56 | 5.05 | 16 |
| 18.41 | 4.82 | 8 |
| 19.61 | 4.52 | 3 |
| 20.10 | 4.41 | 1 |
| 21.38 | 4.15 | 6 |
| 22.95 | 3.87 | 1 |
| 25.52 | 3.49 | 1 |
| 26.42 | 3.37 | 1 |
| 27.75 | 3.21 | 1 |

Note.
Only relative intensities of more than 0% were considered.

### Example 12. Carbon Isotope Characterization of Plant Derived TUDCA

TUDCA from three separate commercial sources presumably derived from animal starting materials, were compared to TUDCA derived from plant derived starting materials, made according to the methods of the current invention, for carbon and isotopic analysis. All analyses performed for elemental and isotopic analysis of carbon were conducted using isotope ratio mass spectrometers that utilize pneumatic type autosamplers, using two different quality control standards. The first standard is a pure chemical that is used to test the instrument linearity and define instrument response for the determination of elemental composition. Methionine (an amino acid) is typically the chemical standard used for this purpose. For each run, the effect of signal on isotopic measurement (linearity) is checked from 200 to 600 ug for carbon. The second standard is used to show measurement stability over the length of the run. This in-house standard is chosen to loosely resemble the matrix of the samples being analyzed. All in-house standards are calibrated periodically against international standards to verify accuracy. Within run isotopic precision for QC standards is 0.2 per mil for carbon. The test results are reported in Table 10.

TABLE 10

| Sample ID | Source | Weight (mg) | % C | δ¹³C v. VPDB |
|---|---|---|---|---|
| 4084-1 | Animal | 0.49 | 58.48 | −14.75 |
| 4084-2 | Animal | 0.572 | 57.98 | −14.78 |
| 4188-1 | Animal | 0.536 | 55.24 | −18.31 |
| 4188-2 | Animal | 0.577 | 57.07 | −18.37 |
| 4077-1 | Plant | 0.499 | 56.41 | −28.95 |
| 4077-2 | Plant | 0.564 | 55.02 | −28.98 |
| 4117-1 | Animal | 0.571 | 57.46 | −17.13 |
| 4117-2 | Animal | 0.52 | 57.65 | −17.09 |

### Example 13. Procedure for Purification of 3,7-DKCA by Way of 3,7-DKCA t-Butylamine Salt

To a 250 mL RBF was taken 3,7-DKCA (20 g) in EtOH (60 mL, 3 vol.) at RT. The mixture was stirred for 15 min to obtain a clear solution. To this solution was added tert-butylamine (TBA, 4.14 g, 1.1 equiv.) in EtOH (40 mL, 2 vol.) over a period of 0.5 h while stirring. A thick slurry was observed within 10 min. and another 20 mL EtOH was added. The suspension was stirred for 2 h at RT, the resulting solid was filtered, the wet cake was washed using cold EtOH (20 mL, 1 vol.) and the product was dried under vacuum to obtain the TBA salt of 3,7-DKCA (3,7-DKCA-TBA, 17.5 g) as an off-white solid.

The 3,7-DKCA-TBA obtained by the foregoing process (34.8 g) was suspended in toluene (174 mL, 5 vol.). The resulting slurry was stirred at 45° C. for 0.5 h and treated with EtOH (522 mL, 15 vol.) at 45° C. The resulting mixture stirred for 20 min to obtain a clear solution. The solvent was evaporated under reduced pressure until ˜7 volumes remained (solid precipitation was observed). Additional EtOH (522 mL, 15 vol.) was added and the solvent was evaporated under reduced pressure until ˜5 volumes remained. The slurry was treated with additional EtOH (174 mL, 5 volumes), stirred at RT for 1 h and the solid was filtered. The wet cake was washed using EtOH (1 vol.) and the solid was dried under vacuum to obtain purified 3,7-DKCA-TBA (22 g) as a white solid.

Melting point=144° C.; Purity by CAD HPLC (area-%)=98% (5α-impurity=not detected); ¹H-NMR (400 MHz, CDCl₃): δ 6.62 (bs, 3H), 2.87 (dd, J=12.4 & 5.6 Hz, 1H), 2.49 (t, J=11.4 Hz, 1H), 0.95-2.40 (m, 27H), 1.31 (s, 9H), 0.93 (d, J=6.4 Hz, 3H), 0.68 (s, 3H).

### Conversion of DKCA-TBA Salt to DKCA

DKCA-TBA salt (20 g) was suspended in water (100 mL). Ethyl acetate (100 mL) was added, followed by 6N HCl (7 mL), leading to a two-phase mixture without any solids. The phases were separated and the organic phase was washed with 1N HCl (20 mL) and then with water (40 mL). The ethyl acetate phase was then concentrated under vacuum to dryness to give a white solid (16 g, 95% yield).

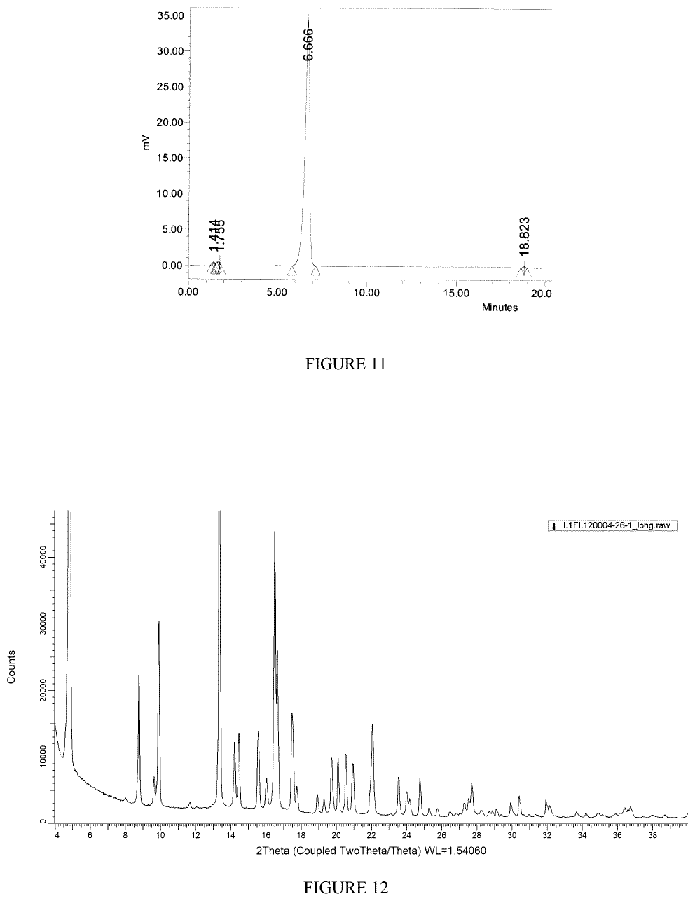

Using validated HPLC methods for measuring the presence of 5α-impurities, the 3,7-DKCA starting material and the final product produced in this Example 13 were subjected to HPLC analysis. The results are depicted in FIG. 10 and FIG. 11, respectively. The 5α-impurity of 3,7-DKCA (RRT 0.88) had an area % of 1.3. The 5α-impurity was not detectable in the final 3,7-DKCA.

### Example 14. Crystalline Salts of 3,7-DKCA

Tert-butylamine, ethylenediamine, and diisopropylamine salts of 3,7-DKCA were crystallized, characterized and scaled up. All three salts showed significant increases in purity, including considerable rejection of the impurity markers of interest. The ethylenediamine salt was observed to be quite polymorphic, with six different forms observed throughout the work. The diisopropylamine salt demonstrated high crystallinity and satisfactory purity results, and considerable mass loss by thermogravimetric analysis (TGA) coincident with an endotherm that had an onset of approximately 86° C. The tert-butylamine salt had high crystallinity, thermal behavior (melting onset at 143.7° C.), and ability to purge impurities, including markers of interest.

Select properties of the products obtained are presented in Table 11.

TABLE 11
Summary of the top results.

| Description | | Results | | | |
|---|---|---|---|---|---|
| Sample | | tert-Butylamine | | Ethylene- | Diisopropyl- |
| | | salt | | diamine | amine |
| | | | | salt | salt |
| XRPD pattern | | 9-A | | 6-D | 10-A |
| DSC onsets (° C.) | | 143.70 | | 192.58 | 83.48, 120.70 |
| TGA mass loss (wt. %) | | 13.15 | | 5.78 | 5.44 |
| Stoichiometry by ¹H NMR | | 1.00:1.06 | | 1.00:0.75 | 1.00:0.88 |
| (FF:CI) | | | | | |
| KF water content (wt. %) | | <0.02 | | 0.86 | 2.40 |
| HPLC chemical purity | | 97.63 | 99.29 | 98.88 | 97.33 |
| (% a/a) | | | | | |
| DVS mass change | | 10.89 | | 19.60 | 2.53 |
| 5-95% RH (wt. %) | | | | | |
| Stability at 40° C. and | | 9-A | | 6-D | 10-A |
| 75% RH | | | | | |
| One-week | EtOAc | 9-A | | 6-D | 10-A |
| form | 2-MeTHF | 9-A | | 6-D + 6-B | 10-A |
| stability in | | | | (trace) | |
| solvents | ACN:water | 9-A | | 6-F | − |
| | (9:1 vol.) | | | | |

Note
CI, counter ion; N/A, not applicable. Hyphen indicates no data were collected.

### Pattern 9-A Scale-Up and XRPD Characterization

Pattern 9-A (tert-Butylamine salt) was scaled up to carry out further characterization. A yield of 123.23 mg (40.0% w/w) with a purity of 99.29% a/a was obtained. The crystallization process was as follows:
1. Weighed 351.4 mg of DKCA into a 20 mL vial
2. Added 4 mL EtOH to the vial
3. Stirred at RT until solid dissolved
4. Weighed 67.1 mg (1.1 equivalents (eq.)) of tert-butylamine into a new 20 mL vial
5. Added 4 mL of EtOH to the vial containing the counter ion
6. Added the counter ion solution to the freeform solution dropwise at RT while stirring at
500 rpm
7. Seeded with 5 mg of Pattern 9-A
8. Heated the solution to 45° C. and stirred for 4 h
9. Cooled down to RT while stirring
10. Filtered and washed with 3 vol. of EtOH
11. Dried in an oven at 50° C. and −29 inHg overnight
   An XRPD peak listing for scaled-up Pattern 9-A is given in Table 12. An XRPD pattern is depicted in FIG. 12.

TABLE 12
XRPD peak list for scaled-up Pattern 9-A.

| Angle | d-spacing | Relative |
|---|---|---|
| (°2θ) | (Å) | intensity |
| 4.83 | 18.28 | 100 |
| 8.77 | 10.07 | 16 |
| 9.68 | 9.13 | 2 |
| 9.90 | 8.93 | 24 |
| 11.67 | 7.58 | 1 |
| 14.08 | 6.29 | 1 |
| 13.35 | 6.63 | 59 |
| 14.22 | 6.23 | 9 |
| 14.46 | 6.12 | 10 |
| 15.56 | 5.69 | 10 |
| 16.03 | 5.52 | 4 |
| 16.49 | 5.37 | 36 |
| 16.65 | 5.32 | 21 |
| 17.51 | 5.06 | 12 |
| 17.76 | 4.99 | 3 |
| 18.92 | 4.69 | 2 |
| 19.30 | 4.60 | 2 |
| 19.73 | 4.50 | 7 |
| 20.10 | 4.41 | 7 |
| 20.54 | 4.32 | 8 |
| 20.95 | 4.24 | 6 |
| 22.05 | 4.03 | 12 |
| 23.53 | 3.78 | 5 |
| 24.00 | 3.70 | 3 |
| 24.18 | 3.68 | 2 |
| 24.75 | 3.59 | 5 |
| 25.28 | 3.52 | 1 |
| 25.75 | 3.46 | 1 |
| 27.26 | 3.27 | 2 |
| 27.52 | 3.24 | 2 |
| 27.71 | 3.22 | 4 |
| 28.27 | 3.15 | 1 |
| 28.70 | 3.11 | 1 |
| 28.88 | 3.09 | 1 |
| 29.12 | 3.06 | 1 |
| 29.93 | 2.98 | 2 |
| 30.40 | 2.94 | 3 |
| 31.97 | 2.80 | 2 |
| 32.14 | 2.78 | 1 |
| 33.66 | 2.66 | 1 |
| 34.20 | 2.62 | 1 |
| 34.91 | 2.57 | 1 |
| 36.43 | 2.46 | 1 |
| 36.74 | 2.44 | 1 |

Note.
The peak cut off used was <1% relative intensity.

### Pattern 6-D Scale-Up and XRPD Characterization

Pattern 6-D Ethylenediamine was scaled up to carry out further characterization. A yield of 207 mg (65.7% w/w) with a purity of 98.88% a/a was obtained. The crystallization process was as follows:
1. Weighed 357.2 mg of DKCA (L1FL120004-2-1) into a 20 mL vial
2. Added 2 mL EtOH to the vial
3. Stirred at RT until solid dissolved
4. Weighed 56.5 mg (1.1 eq.) of ethylenediamine into a new 20 mL vial
5. Added 2 mL of EtOH to the vial containing the counter ion
6. Added the freeform solution to the counter ion solution dropwise at RT while stirring at 500 rpm
7. Heated the solution to 45° C. and stirred for 2 h
8. Evaporated the solvent under nitrogen flow at 45° C.
9. Dried in an oven at 50° C. and −29 inHg for 2 h
10. Added 3 vol. IPA:water (9:1 vol.) to the vial and stirred at RT
11. Heated to 50° C.
12. Cooled down to 5° C. over 12 h
13. Filtered and washed with 3 vol. of IPA:water (9:1 vol.)
14. Dried in an oven at 50° C. and −29 inHg for 3 h
   An XRPD peak list for Pattern 6-D is provided in Table 13. An XRPD pattern is depicted in FIG. 13.

TABLE 13
XRPD peak list for scaled-up Pattern 6-D.

| Angle | d-Spacing | Relative |
|---|---|---|
| (°2θ) | (Å) | intensity (%) |
| 5.81 | 15.19 | 27 |
| 8.69 | 10.17 | 3 |
| 9.95 | 8.88 | 67 |
| 10.92 | 8.10 | 22 |
| 11.60 | 7.62 | 3 |
| 13.08 | 6.76 | 16 |
| 13.78 | 6.42 | 5 |
| 14.59 | 6.07 | 94 |
| 15.19 | 5.83 | 10 |
| 15.38 | 5.76 | 22 |
| 16.03 | 5.52 | 100 |
| 16.51 | 5.37 | 8 |
| 17.40 | 5.09 | 40 |
| 17.77 | 4.99 | 10 |
| 18.11 | 4.89 | 5 |
| 18.89 | 4.69 | 57 |
| 19.21 | 4.62 | 29 |
| 19.94 | 4.45 | 10 |
| 20.27 | 4.38 | 3 |
| 21.04 | 4.22 | 3 |
| 21.32 | 4.16 | 4 |
| 22.10 | 4.02 | 9 |
| 22.27 | 3.99 | 8 |
| 23.45 | 3.79 | 18 |
| 24.04 | 3.70 | 4 |
| 24.24 | 3.67 | 4 |
| 24.90 | 3.57 | 1 |
| 25.11 | 3.54 | 2 |
| 25.60 | 3.48 | 1 |
| 26.00 | 3.42 | 2 |
| 26.23 | 3.39 | 7 |
| 26.82 | 3.32 | 1 |
| 27.42 | 3.25 | 6 |
| 28.10 | 3.17 | 2 |
| 28.33 | 3.15 | 3 |
| 28.82 | 3.10 | 2 |
| 29.69 | 3.01 | 2 |
| 29.88 | 2.99 | 2 |
| 30.24 | 2.95 | 2 |
| 31.57 | 2.83 | 1 |
| 32.36 | 2.76 | 1 |
| 32.59 | 2.75 | 1 |
| 32.87 | 2.72 | 1 |
| 33.35 | 2.68 | 4 |
| 34.73 | 2.58 | 1 |
| 35.18 | 2.55 | 1 |
| 35.54 | 2.52 | 1 |
| 36.07 | 2.49 | 1 |
| 36.70 | 2.45 | 1 |
| 37.24 | 2.41 | 1 |
| 37.52 | 2.39 | 2 |
| 37.83 | 2.38 | 6 |
| 38.22 | 2.35 | 3 |
| 39.42 | 2.28 | 1 |
| 39.80 | 2.26 | 1 |

Note.
The peak cut off used was <1% relative intensity.

### Pattern 10-A Scale-Up and XRDP Characterization

Pattern 10-A (diisopropylamine salt) was scaled up to carry out further characterization. A yield of 175.8 mg (57.1% w/w) with a purity of 97.33% a/a was obtained. The crystallization process was as follows:
1. Weighed 348.2 mg of DKCA (L1FL120004-2-1) into a 20 mL vial
2. Added 2 mL EtOH to the vial
3. Stirred at RT until solid dissolved
4. Weighed 1.1 eq. of diisopropylamine into a new 20 mL vial
5. Added 1 mL of EtOH to the vial containing the counter ion
6. Added the freeform solution to the counter ion solution at RT while stirring at 500 rpm
7. Heated the solution to 45° C. and stirred for 2 h
8. Evaporated the solvent under nitrogen flow at 45° C.
9. Dried in an oven at 50° C. and −29 inHg for 3 h
10. Added 10 vol. MIBK to the vial and stirred at 45° C. for 1 h
11. Cooled down to 25° C.
12. Added 3 vol. heptane dropwise over 15 min
13. Seeded with Pattern 10-A (L1FL120004-7-33)
14. Stirred at RT and 500 rpm over the weekend
15. Filtered and washed with 3 vol. of MIBK:heptane (3:1 vol.)
16. Dried in an oven at 50° C. and −29 inHg for 3 h
   An XRPD peak list for Pattern 10-A is provided in Table 14. An XRPD pattern is depicted in FIG. 14.

TABLE 14
XRPD peak list for scaled-up Pattern 10-A.

| Angle | d-Spacing | Relative intensity |
|---|---|---|
| (°2θ) | (Å) | (%) |
| 5.85 | 15.09 | 43 |
| 6.29 | 14.03 | 4 |
| 9.05 | 9.76 | 41 |
| 11.46 | 7.71 | 57 |
| 11.88 | 7.44 | 21 |
| 12.58 | 7.03 | 26 |
| 13.07 | 6.77 | 63 |
| 13.40 | 6.60 | 3 |
| 13.55 | 6.53 | 5 |
| 14.17 | 6.25 | 16 |
| 14.61 | 6.06 | 100 |
| 14.82 | 5.97 | 4 |
| 16.09 | 5.51 | 36 |
| 17.03 | 5.20 | 2 |
| 17.37 | 5.10 | 3 |
| 17.65 | 5.02 | 8 |
| 18.13 | 4.89 | 68 |
| 18.47 | 4.80 | 32 |
| 18.89 | 4.69 | 3 |
| 19.79 | 4.48 | 9 |
| 20.00 | 4.44 | 10 |
| 20.49 | 4.33 | 15 |
| 21.48 | 4.13 | 2 |
| 23.08 | 3.85 | 23 |
| 23.31 | 3.81 | 10 |
| 23.50 | 3.78 | 5 |
| 23.86 | 3.73 | 4 |
| 24.13 | 3.69 | 12 |
| 24.75 | 3.59 | 2 |
| 25.27 | 3.52 | 2 |
| 25.78 | 3.45 | 6 |
| 26.03 | 3.42 | 6 |
| 26.17 | 3.40 | 6 |
| 26.66 | 3.34 | 1 |
| 26.94 | 3.31 | 1 |
| 27.10 | 3.29 | 2 |
| 27.26 | 3.27 | 3 |
| 27.80 | 3.21 | 2 |
| 28.19 | 3.16 | 4 |
| 28.65 | 3.11 | 2 |
| 29.23 | 3.05 | 3 |
| 29.41 | 3.03 | 4 |
| 30.21 | 2.96 | 2 |
| 30.66 | 2.91 | 3 |
| 31.02 | 2.88 | 3 |
| 31.48 | 2.84 | 1 |
| 31.82 | 2.81 | 2 |
| 32.46 | 2.76 | 4 |
| 32.74 | 2.73 | 3 |
| 34.50 | 2.60 | 1 |
| 34.78 | 2.58 | 2 |
| 34.89 | 2.57 | 2 |
| 35.06 | 2.56 | 3 |
| 35.28 | 2.54 | 2 |
| 37.44 | 2.40 | 2 |

Note.
The peak cut off used was <1% relative intensity.

### Example 15. Purity Characterization

The foregoing examples and general description have illustrated how to obtain TUDCA of remarkable purity, particularly TUDCA derived from non-animal sources. Five of the major impurities implicated in the manufacture of TUDCA, particularly TUDCA from non-animal sources, are controlled as described below:
5-alpha impurities are controlled by hydrogenation with pyridine solvents, and by the formation and crystallization of crystalline salts of DKCA to give undetectable levels of the 5-alpha isomer of DKCA and other 5-alpha impurities;
3-beta impurities are controlled by the use of 3-alpha HSDH (a/k/a ketoreductase) to reduce the 3-ketone, such that no 3-beta impurities are formed or detected in the intermediates or final product TUDCA;
7-alpha impurities are controlled by use of 7-beta-HSDH (a/k/a ketoreductase) to reduce the 7-ketone, such that no 7-alpha impurities are formed or detected in the intermediates or final product TUDCA;
UDCA is controlled and eliminated by not ever using UDCA as an intermediate;
Taurine is controlled by using taurine as a reagent upstream in the process and purging it during the work up and isolation of subsequent intermediates as well as in the final isolation.

To confirm the lack of these impurities, various analyses were undertaken of the final TUDCA produced by the methods of the invention and intermediates thereof. The results are summarized as follows:

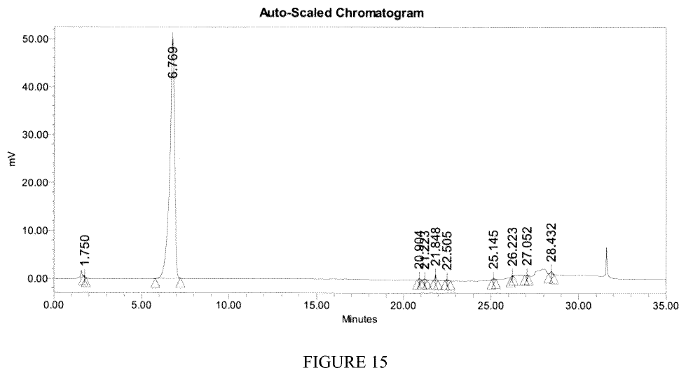

FIG. 15 is an HPLC chromatogram of tert-butylamine salt of 3,7-DKCA produced substantially according to the 3-picoline solvent hydrogenation and tert-butylamine crystallization methods described herein. The dominant peak is tert-butylamine salt of 3,7-DKCA. The 5-alpha impurity of 3,7-DKCA or the tert-butylamine salt of 3,7-DKCA is undetectable.

Table 15 reports purity testing of TUDCA obtained by reducing the 3- and 7-keto groups on 3,7-DKCA using the keto-reductases described herein, and subsequently converting the UDCA to TUDCA using the method described in the examples of WO 2022/039983 (Reid et al.). The 3-picoline solvent hydrogenation and tert-butylamine crystallization methods described herein were also employed. No 3-beta, 5-alpha, or 7-alpha impurities are detected.

TABLE 15
224KAD-A-099

| Impurity | Percentage |
|---|---|
| 3-beta | ND (<0.05%) |
| 5-alpha | ND (<0.05%) |
| 7-alpha | ND (<0.05%) |

ND = none detected

### REFERENCES CITED

Zhang, Y., Werling, U., Ederlmann, W. (2014). Seamless Ligation Cloning Extract (SLiCE) Cloning Method. Methods in Molecular Biology 1116, 235 244.

Throughout this application, various publications are referenced. The disclosures of these publications in their entireties are hereby incorporated by reference into this application in order to more fully describe the state of the art to which this invention pertains. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1-53) (canceled)

54) A compound selected from a taurine conjugate of ursodeoxycholic acid of formula I (TUDCA): 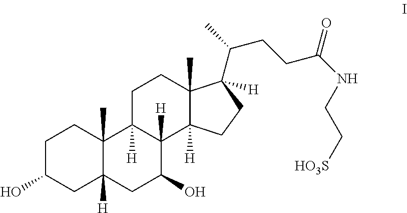
and its salts comprising:
a) a δ¹³C value corresponding to a plant derived molecule, and
b) less than 1.0% of 5α-TUDCA.

55) The compound of claim 54 comprising less than 0.5% of 5α-TUDCA.

56) The compound of claim 54 comprising less than 0.1% of 5α-TUDCA.

57) The compound of claim 54 comprising less than 0.03% of 5α-TUDCA.

58) The compound of claim 54 comprising:
a) less than 1% of any 3β-hydroxysteroids; and
b) less than 1% of any 7α-hydroxysteroids.

59) The compound of claim 54 comprising:
a) less than 1% of UDCA;
b) less than 1% of taurine;
c) less than 1% of any 3β-hydroxysteroids;
d) less than 1% of any 5α-steroids; and
e) less than 1% of any 7α-hydroxysteroids.

60) The compound of claim 54 comprising:
a) less than 0.1% of any 5α-steroids;
b) less than 0.1% of any 3-keto, 7-hydroxysteroids;
c) less than 0.1% of any 3-hydroxy, 7-ketosteroids; and
d) less than 0.1% of TDKCA.

61) The compound of claim 54 comprising:
a) less than 0.1% of any 5α-steroids;
b) less than 0.1% of any 3-keto, 7-hydroxysteroids;
c) less than 0.1% of any 3-hydroxy, 7-ketosteroids;
d) less than 0.1% of TLCA;
e) less than 0.1% of TDKCA; and
f) less than 0.1% of any 3β-hydroxysteroids.

62) The compound of claim 54 comprising:
a) less than 0.1% of UDCA;
b) less than 0.1% of taurine; and
c) less than 0.20% of TCDCA.

63) The compound of claim 54 comprising:
a) less 0.1% of UDCA;
b) less than 0.1% of taurine;
c) less than 0.1% of any 3β-hydroxysteroids;
d) less than 0.1% of any 5α-steroids; and
e) less than 0.1% of any 7α-hydroxysteroids.

64) The compound of claim 54 comprising an XRPD pattern corresponding to Form A TUDCA or Form L TUDCA, optionally:
a) having an X-ray powder diffraction pattern comprising at least one, three, or five peaks, in terms of 2θ, selected from the group consisting of 5.19, 10.31, 10.49, 19.08, 20.83, 22.03, 23.26, 23.58, 24.89, and 31.09±0.2°; or
b) having an X-ray powder diffraction pattern comprising at one or two peaks, in terms of 2θ, selected from the group consisting of 4.59 and 19.61°±0.2°, optionally in combination with one or any combination of 15.11, 17.56, 18.41, and 21.38°±0.2°.

65) The compound of claim 54 selected from the group consisting of arginine TUDCA, lysine TUDCA, and histidine TUDCA, optionally:
a) having an XRPD pattern comprising at least one, three, five, or seven peaks, in terms of 2θ, selected from the group consisting of 11.48, 15.34, 18.43, 19.19, 21.77, 23.08, and 25.29±0.2°;
b) having an XRPD pattern comprising at least one, three, or five peaks, in terms of 2θ, selected from the group consisting of 8.74, 10.38, 12.24, 17.25, and 20.05° 0.2°; or
c) having an XRPD pattern comprising at least one, two, or three in terms of 2θ, selected from the group consisting of 6.76, 9.40, and 12.38°±0.2°.

66) The compound of claim 54 comprising a δ¹³C value of less than −20‰, −22.5‰, or −25% o δ13C relative to VPDB.

67) The compound of claim 54 in an isolated state.

68) The compound of claim 54 comprising less than 3% total impurities selected from starting materials, by-products, intermediates, and degradation products.

69) A pharmaceutical composition comprising the compound of claim 54 and one or more pharmaceutically acceptable excipients.

70) The pharmaceutical composition of claim 69 in the form of a tablet, capsule, or sachet.

71) A method of making a TUDCA pharmaceutical dosage form comprising admixing the compound of claim 54 with one or more pharmaceutically acceptable excipients to form an admixture and processing the admixture into a finished dosage form.

72) The method of claim 71, wherein the processing comprises compressing the admixture into a tablet or filling the admixture into a capsule or sachet.

73) A compound selected from:
a) 3α-Hydroxy-7-oxo-5β-cholanoyltaurine or a salt thereof,
b) 7β-Hydroxy-3-oxo-5β-cholanoyltaurine or a salt thereof,
c) 3,7-Oxo-5β-cholanoyltaurine or a salt thereof;
d) an ethylenediamine salt of 3,7-DKCA;
e) a tert-butylamine salt of 3,7-DKCA; and
f) a diisopropylamine salt of 3,7-DKCA.
